SEQUENCE LISTING (12 sequences)

SEQ ID NO: 1 - DNA, 17 bp, Artificial Sequence
  Synthesized
gacgactgtcttggtgg

SEQ ID NO: 2 - DNA, 17 bp, Artificial Sequence
  Synthesized
atatccaaacaagcctg

SEQ ID NO: 3 - DNA, 20 bp, Artificial Sequence
  Synthesized
ccacgcaaagccaagacgac

SEQ ID NO: 4 - DNA, 20 bp, Artificial Sequence
  Synthesized
gaaaccggcctgctcatcct

SEQ ID NO: 5 - DNA, 20 bp, Artificial Sequence
  Synthesized
atgagacaggaatccgaaca

SEQ ID NO: 6 - DNA, 20 bp, Artificial Sequence
  Synthesized
tcaacgttactgctgtcgaa

SEQ ID NO: 7 - DNA, 20 bp, Artificial Sequence
  Synthesized
tcccgaataaaagcgaagag

SEQ ID NO: 8 - DNA, 20 bp, Artificial Sequence
  Synthesized
aagcttcgggatttatggtg

SEQ ID NO: 9 - DNA, 20 bp, Artificial Sequence
  Synthesized
agaacatcatccctgcatcc

SEQ ID NO: 10 - DNA, 20 bp, Artificial Sequence
  Synthesized
tccaccaccctgttgctgta

SEQ ID NO: 11 - DNA, 21 bp, Artificial Sequence
  Synthesized
ccaagagccgggcccgagtga

SEQ ID NO: 12 - DNA, 20 bp, Artificial Sequence
  Synthesized
aagggcggaggccgagaccg

(19) United States
(12) Patent Application Publication
(10) Pub. No.: US 2020/0208106 A1
(43) Pub. Date: Jul. 2, 2020

(54) Production Method of Multiple Regenerated Hair Follicle Primordia, Production Method of Hair Follicle Tissue-Containing Sheet, Hair Regeneration Kit and Method for Screening Hair Growth promoter or Hair Growth Inhibitor

(71) Applicant: National University Corporation Yokohama National University, Kanagawa (JP)

(72) Inventors: Junji Fukuda, Yokohama-shi, Kanagawa (JP); Tatsuto Kageyama, Yokohama-shi, Kanagawa (JP); Akihiro Shimizu, Yokohama-shi, Kanagawa (JP)

(73) Assignees: National University Corporation Yokohama National University, Kanagawa (JP); Kanagawa Institute of Industrial Science and Technology, Kanagawa (JP)

(21) Appl. No.: 16/640,561

(22) PCT Filed: Aug. 16, 2018
(86) PCT No.: PCT/JP2018/030410

§ 371 Date: Feb. 20, 2020

(30) Foreign Application Priority Data
Aug. 22, 2017 (JP) 2017-159661

(51) Int. Cl. C12N 5/071; G01N 33/50

(52) U.S. Cl. CPC C12N 5/0627; G01N 33/5044; C12N 2502/1352; C12N 2502/00; C12N 2503/02; C12N 2501/113; C12N 2501/00; C12N 2533/54; C12N 2502/28; C12N 2501/115

(57) ABSTRACT

Provided is a manufacturing method for a plurality of regenerated hair follicle germs, including a step including simultaneously inoculating a microwell plate including regularly arranged microwell portions with mesenchymal cells and epithelial cells, and co-culturing the mesenchymal cells and the epithelial cells using a medium containing a fibroblast growth factor while supplying oxygen to the mesenchymal cells and the epithelial cells from at least an upper surface and a bottom surface of the microwell plate, to thereby form hair follicle germs in the microwell portions, the microwell plate being formed of a material having oxygen permeability. Also provided is a kit for hair regeneration, including: a microwell plate including regularly arranged microwell portions; and a fibroblast growth factor, wherein the microwell plate is formed of a material having oxygen permeability.

### TECHNICAL FIELD

The present invention relates to a manufacturing method for a plurality of regenerated hair follicle germs, a manufacturing method for a hair follicle tissue-containing sheet, a kit for hair regeneration, and a screening method for a hair growth-promoting substance or a hair growth-suppressing substance.

This application claims priority from Japanese Patent Application No. 2017-159661, filed on Aug. 22, 2017, the contents of which are incorporated herein by reference in their entirety.

### BACKGROUND ART

In order to establish that hair follicle regenerative medicine is appropriate for clinical application, a regenerated hair follicle needs to have a normal tissue structure, and hair having a hair shaft suited for a transplantation site needs to be formed and grow. Ectodermal appendages including skin appendages, such as hair, generally develop during a fetal period by an interaction between epithelial cells and mesenchymal cells. It is known that a hair follicle, which is one of the ectodermal appendages, experiences growth and regression (hair cycle) repeatedly throughout an individual's life, and during a growth period regeneration of a hair bulb portion is induced by the same molecular mechanism as that in a developmental period of a hair follicle organ. In addition, the regeneration of the hair bulb portion in the hair cycle is considered to be induced by hair papilla cells, which are mesenchymal cells. That is, during the growth period, differentiation of epithelial stem cells of a hair follicle is induced by hair papilla cells, which are mesenchymal cells, to regenerate the hair bulb portion.

Hitherto, for hair follicle regeneration, there has been tried, for example, regeneration of hair follicle variable regions by substitution of mesenchymal cells (hair papilla cells and dermal root sheath cells), neogenesis of hair follicles using mesenchymal cells having a hair follicle-inducing ability, or reconstruction of hair follicles using epithelial cells and mesenchymal cells.

Specifically, for example, there is given a method of forming primitive hair follicle organs, involving inoculating a spheroid vessel, which contains a culture solution supplemented with a Wnt signal activator, with a plurality of cell types of somatic origin (see, for example, Patent Literature 1).

In addition, for example, there is given a method involving arranging a first cell aggregation substantially formed of mesenchymal cells and a second cell aggregation substantially formed of epithelial cells in divided sections within a gel to construct hair follicle germs, inserting a guide, such as a chemical fiber, thereinto, and then transplanting the resultant to regenerate hair follicle organs (see, for example, Patent Literature 2).

In addition, the inventors of the present invention have heretofore developed a manufacturing method for a plurality of regenerated hair follicle germs (see, for example, Patent Literature 3). Specifically, the method includes a step of forming hair follicle germs by inoculating a microwell plate, which includes regularly arranged microwell portions, with mesenchymal cells and epithelial cells, and co-culturing the mesenchymal cells and the epithelial cells while supplying oxygen thereto from at least an upper surface and a bottom surface of the microwell plate.

Meanwhile, it has recently been revealed that a high hair regeneration ability can be achieved by culturing mesenchymal cells using a culture solution containing a platelet rich plasma extract and transplanting the mesenchymal cells to the skin of a mouse with an innate lack of hair growth (see, for example, Non Patent Literature 1).

### CITATION LIST

### Patent Literature

[PTL 1] JP 2015-165823 A
[PTL 2] WO 2012/108069 A1
[PTL 3] WO 2017/073625 A1

### Non Patent Literature

[NPL 1] Xiao S-E., et al., “As a carrier-transporter for hair follicle reconstitution, platelet-rich plasma promotes proliferation and induction of mouse dermal papilla cells.”, Nature, Scientific Reports, 7: 1125, DOI:10.1038/s41598-017-01105-8, 2017.
[NPL 2] Toyoshima, K., et al., “Fully functional hair follicle regeneration through the rearrangement of stem cells and their niches”, Nat. Commun., vol. 3, no. 784, 2012.

### SUMMARY OF INVENTION

### Technical Problem

However, in Non Patent Literature 1, only mesenchymal cells are cultured using a culture solution containing a platelet rich plasma extract, and co-culture of mesenchymal cells and epithelial cells is not investigated. In addition, it is not identified which of the components contained in the platelet rich plasma extract is an effective component.

In addition, as described in Patent Literature 3, the inventors of the present invention have heretofore paid attention to a culture method involving using a culture vessel having excellent oxygen permeability with a view to manufacturing a large amount of hair follicle germs formed of mesenchymal cells and epithelial cells. However, although various medium components to be used for culturing mesenchymal cells or epithelial cells without impairing their respective functions have heretofore been known, there is no knowledge of a specific medium component needed for obtaining a large amount of hair follicle germs having excellent hair regeneration efficiency in the manufacture of hair follicle germs with the above-mentioned culture vessel.

Accordingly, there has been a demand for a method superior to the above-mentioned related-art manufacturing method in that the method enables the manufacture of a plurality of regular and high-density regenerated hair follicle germs having high hair regeneration efficiency and being similar to hair follicle tissues of a mammal.

The present invention has been made in view of the above-mentioned circumstances, and provides a manufacturing method for a plurality of regular and high-density regenerated hair follicle germs of excellent hair regeneration efficiency and similar to hair follicle tissues of a mammal, and a kit for hair regeneration. The present invention also provides a manufacturing method for a hair follicle tissue-containing sheet using the above-mentioned manufacturing method for a plurality of regenerated hair follicle germs, and a screening method for a hair growth-promoting substance or a hair growth-suppressing substance.

### Solution to Problem

That is, the present invention includes the following aspects. A manufacturing method for a plurality of regenerated hair follicle germs according to a first aspect of the present invention is a method including a step including simultaneously inoculating a microwell plate including regularly arranged microwell portions with mesenchymal cells and epithelial cells, and co-culturing the mesenchymal cells and the epithelial cells using a medium containing a fibroblast growth factor while supplying oxygen to the mesenchymal cells and the epithelial cells from at least an upper surface and a bottom surface of the microwell plate, to thereby form hair follicle germs in the microwell portions, the microwell plate being formed of a material having oxygen permeability.

The step of forming hair follicle germs may further include inoculating the microwell plate with cells capable of constructing blood vessels simultaneously with the mesenchymal cells and the epithelial cells.

The cells capable of constructing blood vessels may be vascular endothelial cells.

The fibroblast growth factor may be basic fibroblast growth factor.

A content of the fibroblast growth factor in the medium may be 1 ng/mL or more and 500 ng/mL or less.

The medium may contain platelet rich plasma as the fibroblast growth factor.

A manufacturing method for a hair follicle tissue-containing sheet according to a second aspect of the present invention is a method including a step of transferring a plurality of regenerated hair follicle germs obtained by the manufacturing method for a plurality of regenerated hair follicle germs according to the first aspect to a biocompatible hydrogel in a state in which the plurality of regenerated hair follicle germs are kept in the microwell portions. A density of the microwell portions in the microwell plate may be 20 wells/cm² or more and 500 wells/cm² or less.

The biocompatible hydrogel may be collagen.

A kit for hair regeneration according to a third aspect of the present invention includes: a microwell plate including regularly arranged microwell portions; and a fibroblast growth factor, wherein the microwell plate is formed of a material having oxygen permeability.

The kit for hair regeneration according to the third aspect may further include a medium.

The medium may be one or more kinds selected from the group consisting of a mesenchymal cell growth medium, an epithelial cell growth medium, and a vascular endothelial cell growth medium.

The fibroblast growth factor may be basic fibroblast growth factor.

The kit may include platelet rich plasma as the fibroblast growth factor.

A screening method for a hair growth-promoting substance or a hair growth-suppressing substance according to a fourth aspect of the present invention is a method including: a step 1 of bringing a candidate substance into contact with mesenchymal cells and epithelial cells; a step 2 including inoculating a culture vessel with each of: the mesenchymal cells and the epithelial cells brought into contact with the candidate substance; and as a control, mesenchymal cells and epithelial cells free of contact with the candidate substance, and co-culturing the cells using a medium containing a fibroblast growth factor while supplying oxygen, to thereby form hair follicle germs brought into contact with the candidate substance and control hair follicle germs in the culture vessel; and a step 3 including: judging the candidate substance to be a hair growth-promoting substance when a hair shaft-like structure is formed in the hair follicle germs brought into contact with the candidate substance earlier than in the control hair follicle germs; judging the candidate substance to be a hair growth-suppressing substance when a hair shaft-like structure is formed in the hair follicle germs brought into contact with the candidate substance later than in the control hair follicle germs; and judging the candidate substance to be neither a hair growth-promoting substance nor a hair growth-suppressing substance when a hair shaft-like structure is formed in the hair follicle germs brought into contact with the candidate substance at the same time as in the control germs.

### Advantageous Effects of Invention

According to the manufacturing method for a plurality of regenerated hair follicle germs and the kit for hair regeneration according to the above-mentioned aspects, there are provided a plurality of regular and high-density regenerated hair follicle germs having excellent hair regeneration efficiency and similar to hair follicle tissues of a mammal. In addition, according to the manufacturing method fora hair follicle tissue-containing sheet according to the above-mentioned aspect, there is provided a hair follicle tissue-containing sheet including regular and high-density hair follicle tissues having excellent hair regeneration efficiency. According to the screening method for a hair growth-promoting substance or a hair growth-suppressing substance according to the above-mentioned aspect, screening for a hair growth-promoting substance or a hair growth-suppressing substance is performed in a simple manner.

### BRIEF DESCRIPTION OF DRAWINGS

FIG. 1 is a schematic process diagram for illustrating an example of a manufacturing method for a plurality of regenerated hair follicle germs according to the present invention.

FIG. 2 is a schematic process diagram for illustrating an example of the manufacturing method for a plurality of regenerated hair follicle germs according to the present invention.

FIG. 3 is a schematic process diagram for illustrating an example of a manufacturing method for a hair follicle tissue-containing sheet according to the present invention.

FIG. 4 is a schematic process diagram for illustrating an example of the manufacturing method for a hair follicle tissue-containing sheet according to the present invention.

FIG. 5 is a schematic process diagram for illustrating a production method for a microwell plate in Example 1.

FIG. 6A is a micrograph for showing the state of self-aggregation on the 3rd day of culture of mesenchymal cells and epithelial cells cultured using a medium containing a platelet rich plasma solution (PRPr+) in Example 1. The scale bar represents 100 μm.

FIG. 6B is a micrograph for showing the state of self-aggregation on the 3rd day of culture of mesenchymal cells and epithelial cells cultured using a medium free of a platelet rich plasma solution (PRPr−) in Example 1. The scale bar represents 100 μm.

FIG. 7A is a graph for showing the relative expression amount of Versican on the 3rd day of culture of the mesenchymal cells and epithelial cells cultured under the PRPr+ or PRPr− condition in Example 1. In the graph, the expression amount of Versican under the PRPr+ condition when the expression amount of Versican under the PRPr− condition is defined as 1 is shown.

FIG. 7B is a graph for showing the relative expression amount of Nexin on the 3rd day of culture of the mesenchymal cells and epithelial cells cultured under the PRPr+ or PRPr− condition in Example 1. In the graph, the expression amount of Nexin under the PRPr+ condition when the expression amount of Nexin under the PRPr− condition is defined as 1 is shown.

FIG. 7C is a graph for showing the relative expression amount of Igfbp5 on the 3rd day of culture of the mesenchymal cells and epithelial cells cultured under the PRPr+ or PRPr− condition in Example 1. In the graph, the expression amount of Igfbp5 under the PRPr+ condition when the expression amount of Igfbp5 under the PRPr− condition is defined as 1 is shown.

FIG. 7D is a graph for showing the relative expression amount of Tgβ2 on the 3rd day of culture of the mesenchymal cells and epithelial cells cultured under the PRPr+ or PRPr− condition in Example 1. In the graph, the expression amount of Tgβ2 under the PRPr+ condition when the expression amount of Tgβ2 under the PRPr− condition is defined as 1 is shown.

FIG. 8A is a micrograph for showing the state of a transplantation portion of a nude mouse after 3 weeks from the transplantation of hair follicle germs formed under the PRPr+ condition obtained in Example 1 in Test Example 1. The scale bar represents 1,000 μm.

FIG. 8B is a micrograph for showing the state of a transplantation portion of a nude mouse after 3 weeks from the transplantation of hair follicle germs formed under the PRPr− condition obtained in Example 1 in Test Example 1. The scale bar represents 1,000 μm.

FIG. 9 is a graph for showing the number of regenerated hairs at the transplantation portion of a nude mouse after 3 weeks from the transplantation of the hair follicle germs formed under the PRPr+ or PRPr− condition obtained in Example 1 in Test Example 1.

FIG. 10 includes micrographs for showing hair shaft elongation times and hair shaft-like structures in hair follicle germs in Example 2. On the left side is a micrograph for showing the hair shaft elongation time (12th day of culture) in a hair follicle germ formed under the condition of adding no fibroblast growth factor 2 (FGF2) (FGF2−) and the hair shaft-like structure (arrow) of the hair follicle germ on the 23rd day of culture. Meanwhile, on the right side is a micrograph for showing the hair shaft elongation time (8th day of culture) in a hair follicle germ formed under the condition of adding FGF2 (FGF2+) and the hair shaft-like structure (arrow) of the hair follicle germ on the 10th day of culture. The scale bars each represent 200 μm.

FIG. 11 is a graph for showing the relative expression amounts of Versican and Tgβ2 on the 10th day of culture of the hair follicle germs formed under the FGF2+ or FGF2− condition in Example 2. In the graph, the expression amount of Versican or Tgβ2 under the FGF2+ condition when the expression amount of Versican or Tgβ2 under the FGF2− condition is defined as 1 is shown.

FIG. 12 includes micrographs for showing the states of self-aggregation on the 3rd day of culture of mesenchymal cells and epithelial cells cultured under PRP+(5%, 10%, and 20%) and PRP− (0%) conditions in Reference Example 1. The scale bars each represent 100 μm.

FIG. 13 is a graph for showing the relative expression amount of Versican on the 3rd day of culture of the mesenchymal cells and epithelial cells cultured under the PRP+(5%) or PRP− (0%) condition in Reference Example 1. In the graph, the expression amount of Versican under each condition when the expression amount of Versican under the PRP− condition is defined as 1 is shown.

FIG. 14 is a graph for showing the relative expression amounts of Versican on the 3rd day of culture of mesenchymal cells and epithelial cells cultured under FGF2+(1 ng/mL, 10 ng/mL, and 40 ng/mL) conditions in Example 3. In the graph, the expression amount of Versican (Vcan) under each condition when the expression amount of Versican (Vcan) under the FGF2+(1 ng/mL) condition is defined as 1 is shown.

FIG. 15 is a graph for showing the relative expression amounts of Versican on the 3rd day of culture of mesenchymal cells and epithelial cells cultured under FGF2+(10 ng/mL), FGF2−/PRPr+, and FGF2-/PRPr− conditions in Example 3. In the graph, the expression amount of Versican (Vcan) under each condition when the expression amount of Versican (Vcan) under the FGF2−/PRPr− condition is defined as 1 is shown.

FIG. 16 includes micrographs for showing the states of self-aggregation on the 3rd day of culture of mesenchymal cells and epithelial cells cultured under FGF2+(10 ng/mL and 40 ng/mL), FGF2−/PRPr+, and FGF2−/PRPr− conditions in Example 4. The scale bars each represent 100 μm. The upper images are bright-field images, and the lower images are dark-field images.

FIG. 17 is a graph for showing the relative expression amounts of Versican on the 3rd day of culture of the mesenchymal cells and epithelial cells cultured under the FGF2+(10 ng/mL and 40 ng/mL), FGF2−/PRPr+, and FGF2−/PRPr− conditions in Example 4. In the graph, the expression amount of Versican under each condition when the expression amount of Versican under the FGF2−/PRPr− condition is defined as 1 is shown.

FIG. 18 is a graph for showing the relative expression amounts of Versican and Wnt10b on the 3rd day of culture of mesenchymal cells and epithelial cells cultured under FGF2+(10 ng/mL, 50 ng/mL, and 100 ng/mL) conditions in Example 5.

FIG. 19 includes micrographs for showing the states of transplantation portions of nude mice after 3 weeks from the transplantation of hair follicle germs formed under the FGF2+(10 ng/mL and 100 ng/mL) or FGF2− (0 ng/mL) conditions obtained in Example 5 in Test Example 2.

FIG. 20A is a graph for showing the relative expression amounts of Wnt10b on the 3rd day of culture of mesenchymal cells and epithelial cells cultured under FGF2− (0 ng/mL) or FGF2+(1 ng/mL, 10 ng/mL, and 100 ng/mL) conditions in Example 6.

FIG. 20B is a graph for showing the relative expression amounts of Versican on the 3rd day of culture of mesenchymal cells and epithelial cells cultured under PDGF− (0 ng/mL) or PDGF+(1 ng/mL, 10 ng/mL, and 100 ng/mL) conditions in Example 6.

FIG. 20C is a graph for showing the relative expression amounts of Wnt10b on the 3rd day of culture of the mesenchymal cells and epithelial cells cultured under the PDGF− (0 ng/mL) or PDGF+(1 ng/mL, 10 ng/mL, and 100 ng/mL) conditions in Example 6.

FIG. 20D is a graph for showing the relative expression amounts of Versican on the 3rd day of culture of mesenchymal cells and epithelial cells cultured under VEGF (0 ng/mL) or VEGF (1 ng/mL, 10 ng/mL, and 100 ng/mL) conditions in Example 6.

FIG. 20E is a graph for showing the relative expression amounts of Wnt10b on the 3rd day of culture of the mesenchymal cells and epithelial cells cultured under the VEGF (0 ng/mL) or VEGF (1 ng/mL, 10 ng/mL, and 100 ng/mL) conditions in Example 6.

FIG. 21 is a micrograph for showing the state of a transplantation portion of a nude mouse on the 18th day from the transplantation of hair follicle germs formed under an FGF2+(10 ng/mL) condition obtained in Example 7 in Test Example 3.

FIG. 22 is a micrograph for showing the state of a transplantation portion of a nude mouse on the 14th day from the transplantation of hair follicle germs formed under an FGF2+(10 ng/mL) condition obtained in Example 8 in Test Example 4.

FIG. 23A is a micrograph for showing the state of a transplantation portion of a nude mouse on the 16th day from the transplantation of hair follicle germs formed under an FGF2+(100 ng/mL) condition obtained in Example 9 in Test Example 5.

FIG. 23B is a micrograph for showing the state of a transplantation portion of a nude mouse after 50 days from the transplantation of hair follicle germs formed under the FGF2+(100 ng/mL) condition obtained in Example 9 in Test Example 5.

FIG. 23C is an electron micrograph for showing the surface structure of a regenerated hair in Test Example 5.

FIG. 23D includes micrographs for showing a hair cycle at the transplantation portion in Test Example 5.

FIG. 24 is a micrograph for showing the state of a transplantation portion of a nude mouse on the 30th day from the transplantation of hair follicle germs formed under an FGF2+(100 ng/mL) condition obtained in Example 10 in Test Example 6. The scale bar represents 100 μm.

### DESCRIPTION OF EMBODIMENTS

<<Manufacturing Method for Plurality of Regenerated Hair Follicle Germs>>

A manufacturing method for a plurality of regenerated hair follicle germs according to one embodiment of the present invention includes a step of simultaneously inoculating a microwell plate including regularly arranged microwell portions with mesenchymal cells and epithelial cells, and co-culturing the mesenchymal cells and the epithelial cells using a medium containing a fibroblast growth factor (FGF) while supplying oxygen to the mesenchymal cells and the epithelial cells from at least an upper surface and a bottom surface of the microwell plate, to thereby form hair follicle germs in the microwell portions. In addition, the microwell plate includes regularly arranged microwell portions and is formed of a material having oxygen permeability.

According to the manufacturing method according to this embodiment, through the use of the microwell plate including regularly arranged microwell portions, regular and high-density hair follicle germs similar to hair follicle tissues of a mammal are regenerated. In addition, mesenchymal cells and epithelial cells are brought into contact with each other in a three-dimensional cell aggregation to perform signal transduction at the time of culture, and hence when a medium containing an FGF is used at the time of culture, the FGF is presumed to act on the epithelial cells directly, indirectly via the mesenchymal cells, or through both of these routes to enhance a hair regeneration ability in the hair follicle germs. Accordingly, the formed hair follicle germs achieve excellent hair regeneration efficiency.

As used herein, the term “mesenchymal cells” means cells derived from a mesenchymal tissue or cells obtained by culturing such cells. Examples of the mesenchymal cells include hair papilla cells, dermal root sheath cells, skin mesenchymal cells in a developmental period, and hair follicle mesenchymal cells induced from pluripotent cells (e.g., embryonic stem (ES) cells, embryonic germ (EG) cells, and induced pluripotent stem (iPS) cells).

As used herein, the term “epithelial cells” means cells derived from an epithelial tissue and cells obtained by culturing such cells. Examples of the epithelial cells include cells of the outermost layer of the outer root sheath in the bulge region, epithelial cells of the hair matrix portion, and hair follicle epithelial cells induced from pluripotent cells (e.g., embryonic stem (ES) cells, embryonic germ (EG) cells, and induced pluripotent stem (iPS) cells).

The origin of each of the above-mentioned mesenchymal cells and the above-mentioned epithelial cells is an animal, and is preferably a vertebrate, more preferably a mammal.

Examples of the mammal include, but are not limited to: humans, chimpanzees, and other primates; and domestic animals, pet animals, and experimental animals, such as dogs, cats, rabbits, horses, sheep, goats, cattle, pigs, rats (including nude rats), mice (including nude mice and SCID mice), and guinea pigs.

Of those, the origin of the cells is particularly preferably a human.

As used herein, the term “hair follicle germ” means a tissue from which a hair follicle is derived, and the hair follicle germ is mainly formed of the above-mentioned mesenchymal cells and the above-mentioned epithelial cells. The hair follicle germ is formed in the following sequence. First, the epithelial cells thicken and invaginate toward the mesenchymal cells to engulf a cell aggregate (spheroid) of the mesenchymal cells. Subsequently, the epithelial cells engulfing the spheroid of the mesenchymal cells form a hair matrix germ, and the spheroid of the mesenchymal cells forms a hair papilla having a hair-inducing ability. Thus, a hair follicle germ including the hair matrix germ, the hair papilla, and the like is formed. In the hair follicle germ, the hair papilla provides growth factors to the hair matrix germ and induces the differentiation of the hair matrix germ, and the differentiated cells can form hair.

As used herein, the term “hair follicle” means a skin appendage that is a portion where the epidermis penetrate inwardly in the form of a cylinder and that produces hair.

As used herein, the term “regenerated hair follicle germ” means, for example, a hair follicle germ produced by the manufacturing method according to this embodiment or the like.

As used herein, the term “plurality of regenerated hair follicle germs” means a plurality of the above-mentioned regenerated hair follicle germs in a state of being clustered together. In the manufacturing method according to this embodiment, a plurality of regenerated hair follicle germs in which a plurality of the above-mentioned hair follicle germs are regularly arrayed at a density similar to the density of pores of a mammal are obtained in a simple manner. In addition, each of the hair follicle germs may be differentiated to form a hair follicle in the plurality of regenerated hair follicle germs.

In the related art, a plurality of regenerated hair follicle germs arranged regularly at a high density have been obtained by a method involving culturing spheroids of mesenchymal cells arranged regularly at a high density, and then inoculating the culture with epithelial cells later so that the epithelial cells to surround the spheroids of the mesenchymal cells.

In contrast, in the manufacturing method according to this embodiment, a plurality of regenerated hair follicle germs having excellent hair regeneration efficiency, in which plurality of hair follicle germs are regularly arrayed at a density similar to the density of pores of a mammal, are obtained in a simple manner by simultaneously inoculating a microwell plate with mesenchymal cells and epithelial cells, and co-culturing the cells therein using a medium containing an FGF while supplying oxygen from at least an upper surface and a bottom surface of the microwell plate.

As used herein, the term “regularly” refers to a state in which hair follicle germs are arranged at regular intervals, and it is appropriate that the intervals be similar to intervals between pores in the skin of a mammal. In addition, particularly when the mammal is a primate including a human, specifically, the density similar to the density of pores of the mammal is preferably 20 germs/cm² or more and 500 germs/cm² or less, more preferably 50 germs/cm² or more and 250 germs/cm² or less, still more preferably 100 germs/cm² or more and 200 germs/cm² or less. When the density falls within the above-mentioned range, hair follicle tissues in which the arrangement of normal hair follicle tissues is reproduced more accurately are regenerated.

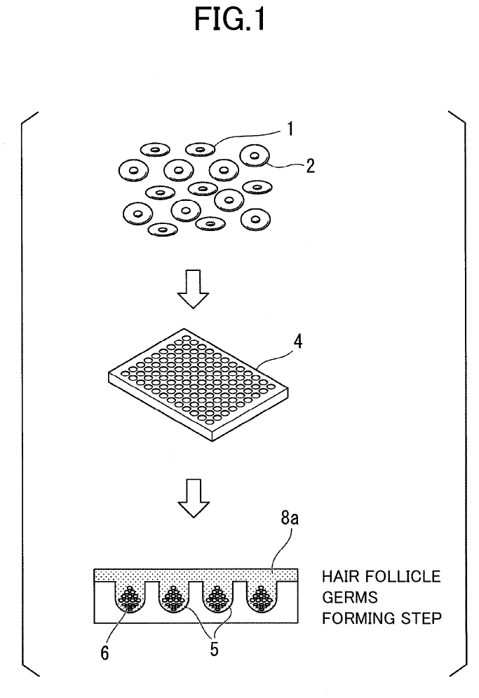

FIG. 1 is a schematic process diagram for illustrating an example of the manufacturing method for a plurality of regenerated hair follicle germs according to the present invention. Details of the step included in the manufacturing method according to this embodiment will be described below with reference to FIG. 1.

[Step of Forming Hair Follicle Germs]

A hair follicle germs forming step in the manufacturing method according to this embodiment is a step of forming hair follicle germs in microwell portions from mesenchymal cells and epithelial cells. Specifically, first, a mixed cell suspension containing mesenchymal cells 1 and epithelial cells 2 is prepared. In this case, a mixing ratio between the mesenchymal cells 1 and the epithelial cells 2, “mesenchymal cells:epithelial cells”, is preferably from 1:2 to 2:1, more preferably from 1:1.5 to 1.5:1, still more preferably 1:1.

Then, the prepared mixed cell suspension is poured into microwell portions of a microwell plate 4. The number of cells for inoculation may be appropriately adjusted depending on the size of microwell portions 5 of the microwell plate 4. At this time, as the number of cells for inoculation increases, the formation efficiency of hair follicle germs becomes higher and the size of each of the hair follicle germs also increases.

Then, the cells are co-cultured using a medium 8a containing an FGF while oxygen is supplied to the cells. A culture time may be set to 1 day or more and 5 days or less (preferably 3 days), and a culture temperature may be set to 25° C. or more and less than 40° C. (preferably 37° C.).

A method of performing culture while supplying oxygen may be, for example, a method involving performing culture while supplying oxygen by direct blowing of oxygen to the microwell plate or the like, or a method involving performing culture using a microwell plate formed of a material having oxygen permeability. Of those, a method involving performing culture using a microwell plate formed of a material having oxygen permeability is preferred because oxygen is supplied to the cells from all surfaces of the microwell plate and the resultant hair follicle germs achieve excellent hair regeneration efficiency.

In the manufacturing method according to this embodiment, the hair follicle germs are formed in the following sequence. First, the epithelial cells 2 thicken and invaginate toward the mesenchymal cells 1 to engulf spheroids of the mesenchymal cells 1. Subsequently, the epithelial cells 2 engulfing the spheroids of the mesenchymal cells 1 form hair matrix germs, and the spheroids of the mesenchymal cells 1 form hair papillae having a hair-inducing ability. Thus, hair follicle germs 6 including the hair matrix germs, the hair papillae, and the like are formed. In the hair follicle germs 6, the hair papillae provide growth factors to the hair matrix germs and induce the differentiation of the hair matrix germs, and the differentiated cells can form hair. Further, in the manufacturing method according to this embodiment, the hair follicle germs may be differentiated to form hair follicles.

The step of forming hair follicle germs may further include inoculating the microwell plate with cells capable of constructing blood vessels simultaneously with the mesenchymal cells and the epithelial cells.

In this case, the hair follicle germs forming step is a step of forming hair follicle germs in the microwell portions by inoculating the microwell plate with a mixed cell suspension containing mesenchymal cells, epithelial cells, and cells capable of constructing blood vessels, and co-culturing the cells using a medium containing an FGF while supplying oxygen to the cells from at least the upper surface and the bottom surface of the microwell plate.

When the mixed cell suspension further contains the cells capable of constructing blood vessels, a plurality of regenerated hair follicle germs each having a capillary structure inside are obtained. When the capillary structure is present in each of the obtained plurality of regenerated hair follicle germs, nutrient components and the like are sufficiently supplied to the inside of each of the hair follicle germs, and hence the hair regeneration efficiency is further improved. Further, when the obtained plurality of regenerated hair follicle germs are used for transplantation, the capillary structure inside each of the hair follicle germs communicates to blood vessels of a transplantation recipient to obtain nutrient components, and hence a high hair follicle-inducing ability is exhibited at a transplantation portion.

As used herein, the term “cells capable of constructing blood vessels” means cells that can construct blood vessels. Examples of the cells capable of constructing blood vessels include vascular endothelial cells and vascular smooth muscle cells, where these cells may be contained alone or in combination thereof.

Of those, the cells capable of constructing blood vessels are preferably vascular endothelial cells.

Examples of the origin of the cells capable of constructing blood vessels include the same ones as those exemplified as the origin of each of the above-mentioned mesenchymal cells and the above-mentioned epithelial cells.

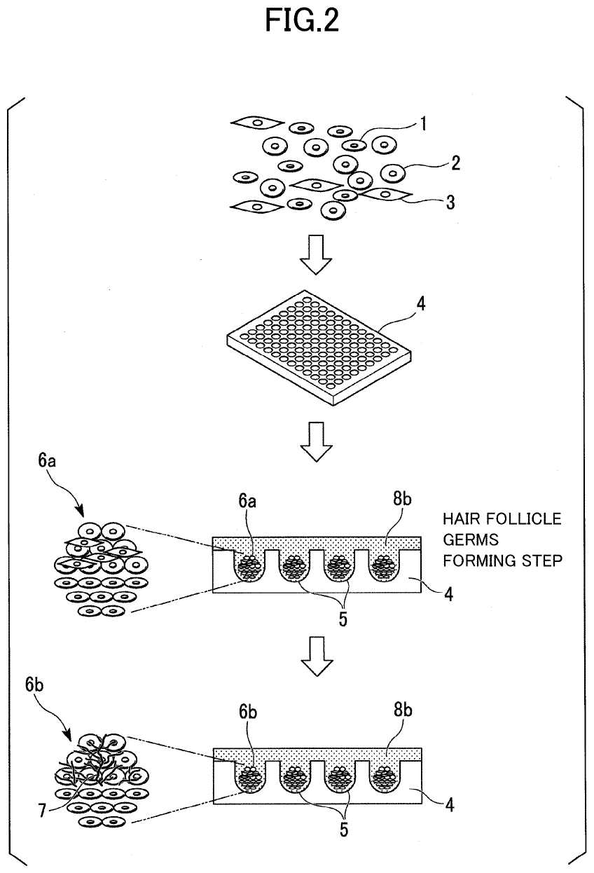

FIG. 2 is a schematic process diagram for illustrating an example of the manufacturing method for a plurality of regenerated hair follicle germs according to the present invention. Details of the step included in the manufacturing method according to this embodiment will be described below with reference to FIG. 2.

When the cells capable of constructing blood vessels are used, in the step of forming hair follicle germs, first, a mixed cell suspension containing the mesenchymal cells 1, the epithelial cells 2, and cells 3 capable of constructing blood vessels is prepared. In this case, a mixing ratio among the mesenchymal cells 1, the epithelial cells 2, and the cells 3 capable of constructing blood vessels, “mesenchymal cells:epithelial cells:cells capable of constructing blood vessels”, is preferably from 2:2:1 to 8:8:1, particularly preferably 4:4:1.

When the mixed cell suspension contains the mesenchymal cells 1, the epithelial cells 2, and the cells 3 capable of constructing blood vessels, the hair follicle germs are formed in the following sequence.

First, the epithelial cells 2 thicken and invaginate toward the mesenchymal cells 1 to engulf spheroids of the mesenchymal cells 1 and the cells 3 capable of constructing blood vessels, with the result that mixed spheroids 6a including the epithelial cells 2, the mesenchymal cells 1, and the cells 3 capable of constructing blood vessels are formed. Subsequently, in the mixed spheroids 6a, the epithelial cells 2 form hair matrix germs, and the spheroids of the mesenchymal cells 1 and the cells 3 capable of constructing blood vessels form hair papillae having a hair-inducing ability. Thus, hair follicle germs 6b including the hair matrix germs, the hair papillae, and the like are formed. In the hair follicle germs 6b, the hair papillae provide growth factors to hair matrix germs and induce the differentiation of the hair matrix germs, and the differentiated cells can form hair. In addition, at this time, on the mesenchymal cell side of each of the hair follicle germs 6b, that is, inside each of the hair papillae, the cells 3 capable of constructing blood vessels construct a capillary structure 7. Further, in the manufacturing method according to this embodiment, the hair follicle germs 6b may be differentiated to form hair follicles.

(Microwell Plate)

The microwell plate 4 to be used in the formation of hair follicle germs is preferably a microwell plate in which a plurality of the microwell portions 5 are regularly arranged. A commercially available microwell plate may be used as the microwell plate 4, or the microwell plate 4 may be produced by, for example, a method to be described later in Example 1. In addition, the density of the microwell portions 5 in the microwell plate 4 is preferably 20 wells/cm² or more and 500 wells/cm² or less, more preferably 50 wells/cm² or more and 250 wells/cm² or less, still more preferably 100 wells/cm² or more and 200 wells/cm² or less. When the density falls within the above-mentioned range, culture is performed in a state in which the hair follicle germs are arranged at a density similar to the density of pores of a mammal (in particular, pores of a primate including a human). As described later, when the regularly arranged high-density hair follicle germs are transplanted to a portion of a recipient animal where hair follicles are defective while the arrangement of the regularly arranged high-density hair follicle germs is maintained, hair follicle tissues in which the arrangement of normal hair follicle tissues is more accurately reproduced are regenerated. In addition, the above-mentioned density of the plurality of regenerated hair follicle germs is considered to be a therapeutically effective amount in the case of using the regenerated hair follicle germs for transplantation.

In addition, the shape of the opening portion of each of the microwell portions is not particularly limited. For example, the shape of the opening portion may be circular, quadrangular, hexagonal, or a line shape. Of those, a circular shape is preferred from the viewpoint that the shape is similar to that of a pore.

The diameter and depth of the opening portion of each of the microwell portions are not particularly limited as long as the size thereof allows for the accommodation and culture of a mixed cell aggregate including the mesenchymal cells and the epithelial cells (hereinafter sometimes referred to as “mixed spheroid”). The diameter of the microwell portion may be similar to the size of a pore of a mammal, and may be, for example, 20 μm or more and 1 mm or less. In addition, the depth of the microwell portion may be 1 mm or less from the viewpoint of fixing a hair follicle tissue-containing sheet to the skin of a recipient animal after transplantation.

The arrangement and sizes of the hair follicle germs to be obtained depend on, for example, the shape of the opening portion, and diameter and depth of each of the microwell portions of the microwell plate, and hence the microwell portions of the microwell plate may be appropriately adjusted depending on the kind of the recipient animal, a transplantation site, and the like.

A material for the microwell plate may be a material suitable for cell culture, and is not particularly limited. Examples of the material include transparent glass and polymer materials. Of those, a polymer material having oxygen permeability is preferred. Specific examples of the polymer material having oxygen permeability include a fluorine resin and a silicon rubber (e.g., poly(dimethylsiloxane): PDMS), where these materials may be used alone or in combination thereof.

As used herein, the term “oxygen permeability” refers to a property of allowing molecular oxygen to permeate the material and reach the interior of each of the microwell portions of the microwell plate. A specific oxygen transmission rate may be about 100 cm³/m²·24 h·atm or more and about 5,000 cm³/m²·24 h·atm or less, about 1,100 cm³/m²·24 h·atm or more and about 3,000 cm³/m²·24 h·atm or less, or about 1,250 cm³/m²·24 h·atm or more and about 2,750 cm³/m²·24 h·atm or less. “24 h” means 24 hours, and “atm” means a unit of atmospheric pressure. That is, the unit “cm³/m²·24 h·atm” represents the volume (cm³) of oxygen to be transmitted per 1 m² in 24 hours under an environment at 1 atm. When a microwell plate formed of a material having an oxygen transmission rate falling within the above-mentioned range is used, a sufficient amount of oxygen is supplied to the mixed spheroids, and hair follicle germs are formed.

(Medium)

FGF

The medium to be used in the manufacturing method according to this embodiment contains an FGF.

In addition, the FGF in the medium may be formed only of the FGF, or may be a mixture containing the FGF as a main component.

Specific examples of the FGF include FGF1 (acidic FGF, aFGF), FGF3, FGF2 (basic FGF, bFGF), FGF3, FGF4, FGF5, FGF6, FGF7, FGF8, FGF9, and FGF10. The medium may contain one kind of those FGFs, or may contain two or more kinds thereof in combination.

Of those, the FGF is preferably FGF2 (basic FGF, bFGF).

When the FGF is a mixture containing the FGF as a main component, a specific example thereof is platelet rich plasma (PRP).

As used herein, the term “platelet rich plasma (PRP)” is a broad term to be used in its general meaning, and refers to plasma having platelets resuspended therein at a concentration higher than that in peripheral blood. The platelet rich plasma (PRP) typically contains platelets at 500,000 platelets/mm³ or more and 1,200,000 platelets/mm³ or less.

In addition, in general, the “platelets” are involved in a hemostatic mechanism and secrete some blood coagulation factors. The platelets also secrete some cytokines involved in wound healing. The cytokines are a group of growth factors to be released from α-granules among granules secreted by the platelets. Examples of the cytokines include FGFs (in particular, FGF2) as main components, and other specific examples include PDGF, TGF-β, VEGF, and PF-4/β-TG.

In this context, “PDGF” refers to a platelet derived growth factor, which has an angiogenic action, that is, an action of forming new blood vessels from existing microvasculature.

“TGF-β” means a transforming growth factor β (Transforming growth factor R).

“VEGF” means a vascular endothelial growth factor.

“PF-4/β-TG” means platelet factor-4/β-thromboglobulin, both of which are released into circulating blood along with the activation of platelets.

The PRP secretes various growth factors including FGFs, and hence when hair follicle germs are cultured using a medium containing the PRP, those growth factors act on the hair follicle germs, with the result that excellent hair regeneration efficiency is achieved.

In addition, the PRP may be prepared by using a method to be described later in Examples. In this embodiment, the PRP to be contained in the medium is preferably PRP in an activated state (PRP-releasate; PRPr). The PRP may be turned to PRPr by being brought into contact with a solution containing calcium ions.

The content of the FGF in the medium is preferably 0.1 ng/mL or more and 500 ng/mL or less, more preferably 1 ng/mL or more and 200 ng/mL or less, still more preferably 1 ng/mL or more and 100 ng/mL or less, particularly preferably 10 ng/mL or more and 100 ng/mL or less. When the content of the FGF in the medium falls within the above-mentioned range, the hair follicle germs to be obtained achieve more excellent hair regeneration efficiency. In particular, in the case where hair follicle germs for autotransplantation are to be manufactured using mesenchymal cells and epithelial cells collected from a patient in need of treatment, when the FGF content is set to a high concentration of about 10 ng/mL or more and about 200 ng/mL or less, excellent hair regeneration efficiency is achieved at a transplantation portion.

In addition, when the mixture containing the FGF as a main component, which is contained in the medium, is PRPr, the content of the PRPr in the medium may be appropriately adjusted depending on the amounts of components, such as the FGF, contained in the PRPr. In general, the PRPr contains about 250 ng/mL of the FGF. More specifically, the content of the PRPr in the medium may be, for example, 1 mass % or more and 15 mass % or less, 3 mass % or more and 10 mass % or less, or 4.5 mass % or more and 6.5 mass % or less. When the content of the PRPr in the medium falls within the above-mentioned range, the content of the FGF is caused to fall within the above-mentioned range, and the hair follicle germs to be obtained achieve more excellent hair regeneration efficiency.

Medium

The medium to be used in the manufacturing method according to this embodiment is not particularly limited, and may be a basal medium containing components required for the survival and growth of cells (an inorganic salt, a carbohydrate, a hormone, an essential amino acid, a non-essential amino acid, and a vitamin) and the like.

The inorganic salt to be contained in the medium serves to help maintain the osmotic pressure equilibrium of cells and to help regulate the membrane potential thereof.

The inorganic salt is not particularly limited, and examples thereof include salts of, for example, calcium, copper, iron, magnesium, potassium, sodium, and zinc. The salt is typically used in the form of any of a chloride, a phosphate, a sulfate, a nitrate, and a bicarbonate.

In general, the osmolality of the medium may be, for example, 200 mOsm/kg or more and 400 mOsm/kg or less, for example, 290 mOsm/kg or more and 350 mOsm/kg or less, for example, 280 mOsm/kg or more and 310 mOsm/kg or less, or for example, 280 mOsm/kg or more and less than 300 mOsm/kg (specifically, 280 mOsm/kg).

The carbohydrate is not particularly limited, and examples thereof include glucose, galactose, maltose, and fructose.

In general, the concentration of the carbohydrate (preferably D-glucose) in the medium is preferably 0.5 g/L or more and 2 g/L or less.

The amino acid is not particularly limited, and examples thereof include L-alanine, L-arginine, L-asparagine, L-aspartic acid, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, L-valine, and combinations thereof.

In general, the concentration of glutamine in the medium is 0.05 g/L or more and 1 g/L or less (typically 0.1 g/L or more and 0.75 g/L or less). The concentration of the amino acid other than glutamine in the medium is 0.001 g/L or more and 1 g/L or less (typically 0.01 g/L or more and 0.15 g/L or less). The amino acid may be derived from synthesis.

The vitamin is not particularly limited, and examples thereof include thiamine (vitamin B1), riboflavin (vitamin B2), niacinamide (vitamin B3), D-pantothenic acid hemicalcium (vitamin B5), pyridoxal/pyridoxamine/pyridoxine (vitamin B6), folic acid (vitamin B9), cyanocobalamin (vitamin B12), ascorbic acid (vitamin C), calciferol (vitamin D2), DL-α-tocopherol (vitamin E), biotin (vitamin H), menadione (vitamin K), choline chloride, and myo-inositol.

The medium may further contain an antibiotic, a serum, a growth factor, or a hormone.

Examples of the antibiotic include antibiotics used for culture of typical animal cells, such as gentamicin, amphotericin, ampicillin, minomycin, kanamycin, penicillin, streptomycin, gentacin, tylosin, and aureomycin. Those antibiotics may be contained alone or in combination thereof.

In general, the concentration of the antibiotic in the medium is not particularly limited, and may be, for example, 0.1 μg/mL or more and 100 μg/mL or less.

Examples of the serum include, but are not limited to, fetal bovine serum/fetal calf serum (FBS/FCS), newborn calf serum (NCS), calf serum (CS), and horse serum (HS).

In general, the concentration of the serum in the medium may be, for example, 2 mass % or more and 10 mass % or less.

The medium may contain a growth factor other than the above-mentioned FGF, and specific examples thereof include, but are not limited to, a cell growth factor and a cell adhesion factor.

More specific examples of the growth factor include an epidermal growth factor (EGF), an insulin-like growth factor-1 (IGF-1), a macrophage-derived growth factor (MDGF), a platelet-derived growth factor (PDGF), and a vascular endothelial growth factor (VEGF). Those growth factors may be contained alone or in combination thereof.

In general, the concentration of the growth factor in the medium is not particularly limited, and may be, for example, 1 ng/mL or more and 10 μg/mL or less.

Examples of the hormone include insulin, glucagon, triiodothyronine, and an adrenocortical hormone (e.g., hydrocortisone). Those hormones may be contained alone or in combination thereof.

In general, the concentration of the hormone in the medium is not particularly limited, and may be, for example, 1 ng/mL or more and 10 μg/mL or less.

In addition, bovine pituitary extract (BPE) may be used as a medium additive containing growth factors and hormones.

Of those, the medium to be used in the manufacturing method according to this embodiment is preferably a medium obtained by mixing a mesenchymal cell growth medium and an epithelial cell growth medium.

A mixing ratio between the mesenchymal cell growth medium and the epithelial cell growth medium, “mesenchymal cell growth medium:epithelial cell growth medium”, is preferably from 1:2 to 2:1, more preferably from 1:1.5 to 1.5 to 1, still more preferably 1:1 in terms of volume ratio.

As the mesenchymal cell growth medium, it is appropriate to use a known basal medium supplemented with any antibiotic and any serum, and as required, any growth factor and any hormone.

Specific examples of the known basal medium (not containing any antibiotic and any serum, and as required, any growth factor and any hormone, and the like) include Dulbecco's Modified Eagle Medium, Minimum Essential Medium (MEM), RPMI-1640, Basal Medium Eagle (BME), DMEM:Nutrient Mixture F-12 (DMEM/F-12), and Glasgow MEM (G-MEM).

In addition, a specific example of the known basal medium containing any antibiotic and any serum, and as required, any growth factor and any hormone is a hair papilla cell growth medium (Follicle Dermal Papilla Cell GrowthMedium; DPCGM) (manufacturedbyPromoCell GmbH).

As the epithelial cell growth medium, it is appropriate to use a known basal medium for epithelial cells containing calcium chloride, free of serum, and supplemented with an epidermal growth factor, and as required, any antibiotic and any hormone.

More specifically, examples of the basal medium for epithelial cells (not containing an epidermal growth factor, and as required, any antibiotic and any hormone) include HuMedia-KB2 (manufactured by Kurabo Industries Ltd.), Keratinocyte Basal Medium 2 (manufactured by PromoCell GmbH), and EpiLife (trademark) Medium (manufactured by Thermo Fisher Scientific).

In addition, examples of the epithelial cell growth medium containing an epidermal growth factor, any antibiotic, and any hormone include HuMedia-KG2 (manufacturedbyKurabo Industries Ltd.) and Keratinocyte Growth Medium 2 (manufactured by PromoCell GmbH).

In the manufacturing method according to this embodiment, when the mixed cell suspension contains mesenchymal cells, epithelial cells, and cells capable of constructing blood vessels, it is preferred to use, as the medium, a medium obtained by mixing a mesenchymal cell growth medium, an epithelial cell growth medium, and a vascular endothelial cell growth medium.

A mixing ratio among the mesenchymal cell growth medium, the epithelial cell growth medium, and the vascular endothelial cell growth medium, “mesenchymal cell growth medium:epithelial cell growth medium:vascular endothelial cell growth medium”, is preferably 1:1:1 in terms of volume ratio.

As the vascular endothelial cell growth medium, it is appropriate to use a known basal medium for vascular endothelial cells supplemented with a vascular endothelial growth factor, and as required, any growth factor, any antibiotic, any serum, and any hormone.

More specifically, examples of the basal medium for vascular endothelial cells (not containing a vascular endothelial growth factor, and as required, any growth factor, any antibiotic, any serum, and any hormone) include EBM-2 Basal Medium (manufactured by Lonza), Endothelial Cell Basal Medium (manufactured by PromoCell GmbH), and Endothelial Cell Basal Medium 2 (manufactured by PromoCell GmbH).

In addition, examples of the vascular endothelial cell growth medium containing a vascular endothelial growth factor, any growth factor, any antibiotic, any serum, and any hormone include EGM2 (manufactured by Lonza), Endothelial Cell Growth Medium (manufactured by PromoCell GmbH), and Endothelial Cell GrowthMedium (manufactured by PromoCell GmbH).

<<Manufacturing Method for Hair Follicle Tissue-Containing Sheet>>

A manufacturing method for a hair follicle tissue-containing sheet according to one embodiment of the present invention is a method including a step of transferring a plurality of regenerated hair follicle germs obtained by the above-mentioned manufacturing method for a plurality of regenerated hair follicle germs to a biocompatible hydrogel in a state in which the plurality of regenerated hair follicle germs are kept in the microwell portions.

According to the manufacturing method according to this embodiment, a regular and high-density hair follicle tissue-containing sheet is obtained in a simple manner. In addition, hair follicle tissues in the obtained hair follicle tissue-containing sheet have excellent hair regeneration efficiency.

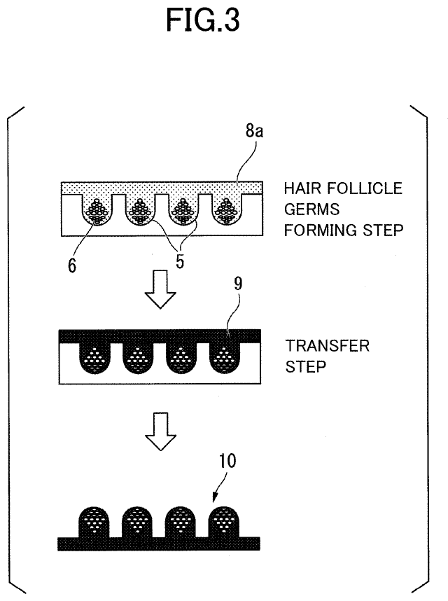

FIG. 3 is a schematic process diagram for illustrating an example of the manufacturing method for a hair follicle tissue-containing sheet according to the present invention. Details of the step included in the manufacturing method according to this embodiment will be described below with reference to FIG. 3.

[Transfer Step]

The transfer step in the manufacturing method according to this embodiment is a step of transferring hair follicle germs formed in the microwell portions of the microwell plate to a biocompatible hydrogel while the hair follicle germs are kept in the microwell portions.

In addition, a method of forming the hair follicle germs to be used in the transfer step is, for example, a method similar to the method described in the above-mentioned manufacturing method for a plurality of regenerated hair follicle germs.

Specifically, in the transfer step, the medium 8a in each of the microwell portions 5 is removed, a solution containing a biocompatible hydrogel 9 is added, and the biocompatible hydrogel 9 is gelated. The concentration of the biocompatible hydrogel 9 in the solution may be appropriately adjusted depending on the required hardness of the gel. In addition, a period of time for the gelation may also be appropriately adjusted depending on the required hardness of the gel. Conditions such as a gelation temperature are not particularly limited, and there is given, for example, a method involving performing culture in a CO₂ incubator at 37° C.

Then, the gelated biocompatible hydrogel 9 containing the hair follicle germs 6 is removed from the microwell plate. Thus, a hair follicle tissue-containing sheet 10 is obtained.

In the obtained hair follicle tissue-containing sheet 10, the hair follicle germs 6 (or hair follicles) are preferably arranged on the biocompatible hydrogel 9 regularly and at a density similar to the density of pores of a mammal. The term “regularly” refers to a state in which the hair follicle germs 6 are arranged at regular intervals, and it is appropriate that the intervals be similar to intervals between pores in the skin of a mammal. In addition, particularly when the mammal is a primate including a human, specifically, the density similar to the density of pores of the mammal is preferably 20 germs/cm² or more and 500 germs/cm² or less, more preferably 50 germs/cm² or more and 250 germs/cm² or less, still more preferably 100 germs/cm² or more and 200 germs/cm² or less. When the density falls within the above-mentioned range, hair follicle tissues in which the arrangement of normal hair follicle tissues is more accurately reproduced are regenerated. In addition, the above-mentioned density of the hair follicle germs in the hair follicle tissue-containing sheet is considered to be a therapeutically effective amount in the case of using the hair follicle tissue-containing sheet for transplantation.

In the transfer step, hair follicle germs each having a capillary structure inside may be used.

In this case, the transfer step is a step of transferring hair follicle germs, each having a capillary structure inside, to a biocompatible hydrogel.

When the hair follicle germs each having a capillary structure inside are used, the resultant hair follicle tissue-containing sheet has a capillary structure inside each of the hair follicle germs. Accordingly, when the hair follicle tissue-containing sheet is used for transplantation, the capillary structure inside each of the hair follicle germs communicates to blood vessels of a transplantation recipient to obtain nutrient components, and hence a high hair follicle-inducing ability is exhibited at a transplantation portion.

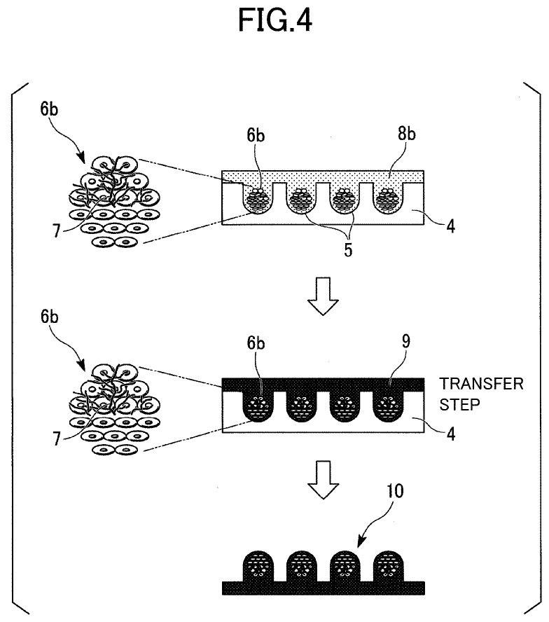

FIG. 4 is a schematic process diagram for illustrating an example of the manufacturing method for a hair follicle tissue-containing sheet according to the present invention. Details of the step included in the manufacturing method according to this embodiment will be described below with reference to FIG. 4.

When the hair follicle germs, each having a capillary structure inside are used, the transfer step is performed in the following sequence.

First, a medium 8b in each of the microwell portions 5 is removed, a solution containing the biocompatible hydrogel 9 is added, and the biocompatible hydrogel 9 is gelated. The concentration of the biocompatible hydrogel 9 in the solution may be appropriately adjusted depending on the required hardness of the gel. In addition, a period of time for the gelation may also be appropriately adjusted depending on the required hardness of the gel. Conditions such as a gelation temperature are not particularly limited, and there is given, for example, a method involving performing culture in a CO₂ incubator at 37° C.

Then, the gelated biocompatible hydrogel 9 containing the hair follicle germs 6b is removed from the microwell plate. Thus, the hair follicle tissue-containing sheet 10 is obtained.

In the obtained hair follicle tissue-containing sheet 10, the hair follicle germs 6b (or hair follicles) are preferably arranged on the biocompatible hydrogel 9 regularly and at a density similar to the density of pores of a mammal. The term “regularly” refers to a state in which the hair follicle germs 6b are arranged at regular intervals, and it is appropriate that the intervals be similar to intervals between pores in the skin of a mammal. In addition, the density similar to the density of pores of a mammal is the same as the density described above.

(Biocompatible Hydrogel)

As used herein, the term “biocompatible hydrogel” refers to a gel having biocompatibility, and means a substance in which a polymer forms a network structure by chemical bonding and a large amount of water is retained in the network. More specifically, the term refers to a substance obtained by gelating a polymer derived from a natural substance or an artificial material such as a synthetic polymer, through the introduction of cross-links thereinto.

An example of the polymer derived from a natural substance is an extracellular matrix component that gelates. Examples of the extracellular matrix component that gelates may include collagen (e.g., type I, type II, type III, type V, and type XI), a basal membrane component (product name: Matrigel) reconstructed from a mouse EHS tumor extract (including type IV collagen, laminin, heparan sulfate proteoglycan, and the like), fibrin, glycosaminoglycan, hyaluronic acid, and proteoglycan. As other polymers derived from natural substances, gelatin, agar, agarose, and the like may also be used. The hydrogel may be produced by selecting conditions optimal for the gelation including a component such as a salt, the concentration thereof, and a pH. Those polymers derived from natural substances may be used alone or in combination thereof.

Examples of the synthetic polymer include polyacrylamide, polyvinyl alcohol, methyl cellulose, polyethylene oxide, and poly(II-hydroxyethylmethacrylate)/polycaprolactone. Those synthetic polymers may be used alone or in combination thereof.

Of those, the biocompatible hydrogel is preferably a polymer derived from a natural substance, more preferably an extracellular matrix component that gelates, still more preferably collagen (in particular, type I collagen). When the biocompatible hydrogel contains collagen, the composition thereof becomes closer to that of the skin, and high hair follicle regeneration efficiency is achieved.

The solution containing the biocompatible hydrogel may contain a serum-free medium, such as Ham's Nutrient Mixtures F-10 or Ham's Nutrient Mixtures F-12, a buffer solution for reconstructing a biocompatible hydrogel (e.g., a buffer solution formed of sodium hydroxide, sodium hydrogen carbonate, and HEPES-Buffer), and the like.

In the manufacturing method according to this embodiment, at the time of the gelation of the biocompatible hydrogel, in order to reinforce the strength of the gel, a support may be incorporated.

A material for the support is not particularly limited as long as the material facilitates the connection between the epithelial cell portion of the hair follicle germs and the epithelial cells of a recipient animal after transplantation. Specific examples of the material for the support include: chemical fibers, such as a fiber made of a polymer such as nylon or a synthetic or natural bioabsorbable polymer, a metal fiber of stainless steel or the like, a carbon fiber, and a glass fiber; and a natural animal fiber (e.g., hair derived from a biological body) or a natural plant fiber. More specific examples of the material for the support include nylon thread and stainless-steel wire.

The diameter and length of the support may be appropriately designed depending on a portion to be subjected to regeneration. The diameter may be, for example, 5 μm or more and 100 μm or less, or 20 μm or more and 50 μm or less. In addition, the length may be, for example, 1 mm or more and 10 mm or less, or 4 mm or more and 6 mm or less.

<<Transplantation Method for Plurality of Regenerated Hair Follicle Germs>>

A transplantation method for a plurality of regenerated hair follicle germs according to one embodiment of the present invention is a method including a step of transplanting a plurality of hair follicle germs obtained by the above-mentioned manufacturing method for a plurality of regenerated hair follicle germs to a portion of a recipient animal where hair follicles are defective while maintaining the regular arrangement of the microwell portions.

According to the transplantation method according to this embodiment, regular and high-density hair follicle tissues are regenerated in a simple manner. In addition, the plurality of regenerated hair follicle germs to be used in the transplantation method according to this embodiment have excellent hair regeneration efficiency. Further, in the transplantation method according to this embodiment, when a plurality of regenerated hair follicle germs each having a capillary structure inside are used, after transplantation, the capillary structure inside each of the hair follicle germs communicates to blood vessels of the recipient animal (transplantation recipient) to obtain nutrient components, and hence a high hair follicle-inducing ability is exhibited at the transplantation portion.

[Transplantation Step]

The hair follicle germs are, for example, aspirated using a multichannel pipette having a plurality of tips, needles, or nozzles that are regularly arranged in the same manner as the above-mentioned microwell portions. Then, the hair follicle germs are transplanted to a portion of a recipient animal where hair follicles are defective, with their regular arrangement being maintained. When the regular arrangement is maintained, hair follicle tissues in which the arrangement of normal hair follicle tissues is more accurately reproduced are regenerated. The multichannel pipette may be a manual one or a fully automatic one.

The recipient animal is preferably any of various mammals including humans or non-human animals, more preferably a human.

Herein, the “multichannel pipette” is not particularly limited as long as: the pipette has a plurality of tips, needles, or nozzles at an end thereof; the tips, the needles, or the nozzles are regularly arranged in the same manner as the above-mentioned microwell portions; and the pipette can aspirate and discharge hair follicle germs. A material therefor is not particularly limited as long as the material is not harmful to cells. In addition, an aperture at an end of each of the tips, needles, or nozzles to be mounted on the multichannel pipette is not particularly limited as long as the size of the aperture is such that the tips, the needles, or the nozzles are inserted into the microwell portions of the microwell plate.

In addition, a transplantation depth may be appropriately changed depending on a site to be subjected to regeneration. The transplantation depth may be, for example, 0.05 mm or more and 5 mm or less, for example, 0.1 mm or more and 1 mm or less, or for example, 0.3 mm or more and 0.5 mm or less.

In addition, with regard to the transplantation site, the hair follicle germs are preferably transplanted into the dermal layer of the recipient animal, and are more preferably transplanted to a site above a boundary surface between the dermis and the subcutaneous tissue because the efficiency of each of hair follicle formation and subsequent hair regeneration is excellent. In addition, it is preferred to adjust the transplantation depth so that the upper end portions of the epithelial cell components of the hair follicle germs are exposed at the upper end portion of the incision for transplantation because continuity with the epithelial cells of the recipient animal is further enhanced.

<<Transplantation Method for Hair Follicle Tissue-Containing Sheet>>

The hair follicle tissue-containing sheet according to this embodiment may be transplanted to a site of interest by a method known to a person skilled in the art. For example, the hair follicle tissue-containing sheet may be transplanted using: Shapiro's hair transplant surgery; hair transplant using a Choi's hair transplanter; or an implanter utilizing air pressure. Shapiro's hair transplant surgery is a method involving making an incision for transplantation at a transplantation site with a microscalpel or the like and then performing transplantation using tweezers.

The size of the hair follicle tissue-containing sheet according to this embodiment is appropriately adjusted in consideration of, for example, the age, sex, and symptoms of the recipient animal, a treatment site, and a treatment time.

In addition, examples of the transplantation depth and the transplantation site include the same ones as those described in the above-mentioned transplantation method for a plurality of regenerated hair follicle germs.

The hair follicle tissue-containing sheet according to this embodiment may be fixed to the transplantation site using a tape or band for skin joining, a suture, or the like.

When the hair follicle tissue-containing sheet according to this embodiment includes the above-mentioned support, the support may be removed from the transplantation site after continuity between the epithelial cells of the recipient animal and a portion derived from the epithelial cells of the hair follicle germs has been secured after awhile following the transplantation of the regenerated hair follicle germs. The time of the removal may be appropriately set depending on the state after the transplantation, but for example, it is preferred that the support be removed from the transplantation site 3 days or more and 7 days or less after the transplantation. Alternatively, the support may be left until the support is spontaneously removed from the transplantation site. A support made of a bioabsorbable material may be left until the support is spontaneously removed from the transplantation site, decomposed, or absorbed.

In addition, when the hair follicle tissue-containing sheet according to this embodiment includes the above-mentioned support, the cells derived from the epithelial cells of the hair follicle germs grow along the support. As a result, the continuity between the epithelial cells of the recipient animal and the epithelial cells of the hair follicle germs after transplantation is improved. In particular, when the support is kept in the exterior of the epidermis at the transplantation site, the epithelial cells of the recipient animal grow toward the interior of the transplantation site along the support so as to eliminate foreign matter. Accordingly, the continuity is further improved. Further, the formation of hair follicles in an intended direction is promoted. As a result, the efficiency of hair regeneration from the hair follicle germs is improved, and besides, the direction of hair growth is controlled.

<<Treatment Method for Regenerating Hair Follicle Tissues>>

According to one embodiment of the present invention, there is provided a therapeutic agent for hair follicle regeneration, including a therapeutically effective amount of a plurality of regenerated hair follicle germs obtained by the above-mentioned manufacturing method.

In addition, according to one embodiment of the present invention, there is provided a therapeutic agent for hair follicle regeneration, including a therapeutically effective amount of a hair follicle tissue-containing sheet obtained by the above-mentioned manufacturing method.

In addition, according to one embodiment of the present invention, there is provided a pharmaceutical composition, including the therapeutic agent for hair follicle regeneration.

In addition, according to one embodiment of the present invention, there is provided a use of a plurality of regenerated hair follicle germs obtained by the above-mentioned manufacturing method, for manufacturing a pharmaceutical composition including the therapeutic agent for hair follicle regeneration.

In addition, according to one embodiment of the present invention, there is provided a use of a hair follicle tissue-containing sheet obtained by the above-mentioned manufacturing method, for manufacturing a pharmaceutical composition including the therapeutic agent for hair follicle regeneration.

In addition, according to one embodiment of the present invention, there is provided a method of treating a defective hair site, such as an epidermis defect or hair loss, caused by a disease, an accident, or the like, the method including transplanting an effective amount of a plurality of regenerated hair follicle germs obtained by the above-mentioned manufacturing method to a patient in need of treatment.

In addition, according to one embodiment of the present invention, there is provided a method of treating a defective hair site, such as an epidermis defect or hair loss, caused by a disease, an accident, or the like, including transplanting an effective amount of a hair follicle tissue-containing sheet obtained by the above-mentioned manufacturing method to a patient in need of treatment.

In the treatment method for regenerating hair follicle tissues according to this embodiment, a tissue to be subjected to transplantation is not particularly limited as long as the tissue is the epidermis of the body in which the regeneration of hair follicles and the regeneration of hair are desired. An example thereof is a scalp.

In addition, the disease to which the present invention is applicable is any disease associated with hair loss, and examples thereof include, but are not limited to, androgenetic alopecia (AGA), female androgenetic alopecia (FAGA), postpartum alopecia, diffuse alopecia, seborrheic alopecia, alopecia pityroides, traction alopecia, alopecia causedbymetabolic disorders, pressure alopecia, alopecia areata, neurotic alopecia, hair-pulling disorder, alopecia universalis, and symptomatic alopecia.

A subject to be treated is not particularly limited, and is preferably a mammal. Examples of the mammal include primates (e.g., humans, monkeys, or chimpanzees), rodents (e.g., mice, rats, guinea pigs, or hamsters), ungulates (e.g., cattle, horses, sheep, goats, or pigs), and pet animals (e.g., dogs, cats, or rabbits). Of those, a human is preferred.

<<Kit for Hair Regeneration>>

A kit for hair regeneration according to one embodiment of the present invention includes a microwell plate and an FGF. In addition, the microwell plate includes regularly arranged microwell portions and is formed of a material having oxygen permeability.

According to the kit for hair regeneration according to this embodiment, through the use of the microwell plate including regularly arranged microwell portions, regular and high-density hair follicle germs similar to hair follicle tissues of a mammal are regenerated. In addition, when a medium containing the FGF is used at the time of culture, the formed hair follicle germs achieve excellent hair regeneration efficiency.

Examples of the microwell plate and FGF included in the kit for hair regeneration according to this embodiment include the same ones as those exemplified in the above-mentioned manufacturing method for a plurality of regenerated hair follicle germs.

The kit for hair regeneration according to this embodiment may further include a medium.

Examples of the medium include the same ones as those exemplified in the above-mentioned manufacturing method for a plurality of regenerated hair follicle germs.

Of those, the medium included in the kit for hair regeneration according to this embodiment is preferably one or more kinds selected from the group consisting of a mesenchymal cell growth medium, an epithelial cell growth medium, and a vascular endothelial cell growth medium.

When only mesenchymal cells and epithelial cells are used for manufacturing a plurality of regenerated hair follicle germs through the use of the kit for hair regeneration according to this embodiment, the mesenchymal cell growth medium and the epithelial cell growth medium are preferred.

When mesenchymal cells, epithelial cells, and cells capable of constructing blood vessels are used for manufacturing a plurality of regenerated hair follicle germs through the use of the kit for hair regeneration according to this embodiment, the mesenchymal cell growth medium, the epithelial cell growth medium, and the vascular endothelial cell growth medium are preferred.

Examples of the mesenchymal cell growth medium, the epithelial cell growth medium, and the vascular endothelial cell growth medium include the same ones as those exemplified in the above-mentioned manufacturing method for a plurality of regenerated hair follicle germs.

The kit for hair regeneration according to this embodiment may further include a biocompatible hydrogel. When the kit includes the biocompatible hydrogel, a hair follicle tissue-containing sheet can be manufactured.

Examples of the biocompatible hydrogel include the same ones as those exemplified in the above-mentioned manufacturing method for a hair follicle tissue-containing sheet.

The kit for hair regeneration according to this embodiment may further include an instrument to be used for transplanting an obtained plurality of regenerated hair follicle germs or hair follicle tissue-containing sheet.

Specific examples of the instrument include, but are not limited to, tweezers and a multichannel pipette.

The kit for hair regeneration according to this embodiment may further include a support.

Examples of the support include the same ones as those exemplified in the above-mentioned manufacturing method for a hair follicle tissue-containing sheet.

<<Screening Method for Hair Growth-Promoting Substance or Hair Growth-Suppressing Substance>>

A screening method for a hair growth-promoting substance or a hair growth-suppressing substance according to one embodiment of the present invention is a method including the following steps 1 to 3.

Step 1: A step of bringing a candidate substance into contact with mesenchymal cells and epithelial cells

Step 2: A step including inoculating a culture vessel with each of: the mesenchymal cells and the epithelial cells brought into contact with the candidate substance; and as a control, mesenchymal cells and epithelial cells free of contact with the candidate substance, and co-culturing the cells using a medium containing a fibroblast growth factor while supplying oxygen, to thereby form hair follicle germs brought into contact with the candidate substance and control hair follicle germs in the culture vessel

Step 3: A step of: judging the candidate substance to be a hair growth-promoting substance when a hair shaft-like structure is formed in the hair follicle germs brought into contact with the candidate substance earlier than in the control hair follicle germs; judging the candidate substance to be a hair growth-suppressing substance when a hair shaft-like structure is formed in the hair follicle germs brought into contact with the candidate substance later than in the control hair follicle germs; and judging the candidate substance to be neither the hair growth-promoting substance nor the hair growth-suppressing substance when a hair shaft-like structure is formed in the hair follicle germs brought into contact with the candidate substance at the same time as in the control germs.

According to the screening method according to this embodiment, screening for a hair growth-promoting substance or a hair growth-suppressing substance is performed in a simple manner through, for example, visual or microscopic observation.

Details of each of the steps included in the screening method according to this embodiment will be described below.

[Step 1]

The step 1 is a step of bringing a candidate substance into contact with mesenchymal cells and epithelial cells.

Examples of the mesenchymal cells and the epithelial cells include the same ones as those exemplified in the above-mentioned manufacturing method for a plurality of regenerated hair follicle germs.

In addition to the mesenchymal cells and the epithelial cells, cells capable of constructing blood vessels may be further simultaneously brought into contact with the candidate substance.

Examples of the cells capable of constructing blood vessels include the same ones as those exemplified in the above-mentioned manufacturing method for a plurality of regenerated hair follicle germs.

In addition, herein, the “candidate substance” encompasses, for example, organic or inorganic compounds (in particular, low-molecular-weight compounds), proteins, and peptides.

A contact time is not particularly limited, and the step 2 and the step 3, which are performed after the step 1, may also be performed in the presence of the candidate substance.

Contact conditions are not particularly limited, and may be conditions under which the cells in a mixed cell suspension survive. Specific examples thereof include a 5% CO₂ environment at a temperature of 25° C. or more and less than 40° C. (preferably 37° C.).

[Step 2]

The step 2 is a step of forming hair follicle germs brought into contact with the candidate substance and controlling hair follicle germs in a culture vessel.

The step 2 is specifically described. First, a mixed cell suspension of the mesenchymal cells and the epithelial cells brought into contact with the candidate substance is poured into the culture vessel. In addition, as a control, a mixed cell suspension of mesenchymal cells and epithelial cells free of contact with the candidate substance is also prepared and poured into the culture vessel.

A material for the culture vessel may be a material suitable for cell culture, and is not particularly limited. Examples thereof include transparent glass and polymer materials. Of those, a polymer material having oxygen permeability is preferred because hair follicle germs are formed in a shorter period of time. Specific examples of the polymer material having oxygen permeability include a fluorine resin and a silicon rubber (e.g., poly(dimethylsiloxane) (PDMS)). Those materials may be used alone or in combination thereof.

Then, the inoculated mixed cells are co-cultured using a medium containing an FGF while oxygen is supplied to the cells. A culture time may be 1 day or more and 14 days or less (preferably about 8 days), and a culture temperature may be 25° C. or more and less than 40° C. (preferably 37° C.).

As described later in Examples, irrespective of the presence or absence of the addition of the FGF, hair follicle germs are formed after about 3 days of culture. When the culture is further continued, it takes about 12 days from the start of the culture for hair follicle germs using a medium free of the FGF to forma hair shaft-like structure. In contrast, hair follicle germs using a medium containing the FGF form a hair shaft-like structure in as short a period of time as about 8 days from the start of culture. In addition, it has been found for the first time by the inventors of the present invention that hair follicle germs form a hair shaft-like structure in the in-vitro test system such as the culture vessel.

Examples of the FGF and medium to be used include the same ones as those exemplified in the above-mentioned manufacturing method for a plurality of regenerated hair follicle germs. Of those, the FGF is preferably FGF2 (basic FGF, bFGF).

[Step 3]

The step 3 is a step of judging the candidate substance to be a hair growth-promoting substance or a hair growth-suppressing substance from the formation state of a hair shaft-like structure in the hair follicle germs.

A comparison to the time at which the hair shaft-like structure is formed in hair follicle germs free of contact with the candidate substance (hereinafter sometimes referred to as “control hair follicle germs”) allows the candidate substance to be judged to be a hair growth-promoting substance or a hair growth-suppressing substance. Specifically, when the hair shaft-like structure is formed in the hair follicle germs brought into contact with the candidate substance earlier than in the control hair follicle germs, the candidate substance may be judged to be a hair growth-promoting substance. Meanwhile, when the hair shaft-like structure is formed in the hair follicle germs brought into contact with the candidate substance later than in the control hair follicle germs, or no hair shaft-like structure is formed therein, the candidate substance may be judged to be a hair growth-suppressing substance. In addition, when the hair shaft-like structure is formed in the hair follicle germs brought into contact with the candidate substance in about the same number of culture days as in the control hair follicle germs, the candidate substance may be judged to have neither a hair growth-promoting action nor a hair growth-suppressing action.

In addition, when the control hair follicle germs are not produced, a judgment may be made on the basis of the number of culture days. Specifically, hair follicle germs at 3 days or more and 14 days or less from the start of culture are subjected to, for example, visual or microscopic observation to confirm the presence or absence of the formation of the hair shaft-like structure. When the time at which the hair shaft-like structure is formed is 3 days or more and less than 8 days from the start of culture, the candidate substance may be judged to be a hair growth-promoting substance. Meanwhile, when the time at which the hair shaft-like structure is formed is 9 days or more and 14 days or less from the start of culture or no hair shaft-like structure is formed, the candidate substance may be judged to be a hair growth-suppressing substance. In addition, when the time at which the hair shaft-like structure is formed is about 8 days from the start of culture, the candidate substance may be judged to have neither a hair growth-promoting action nor a hair growth-suppressing action.

Further, the expression amount of a marker gene associated with hair growth in hair follicle germs (e.g., Versican gene, Nexin gene, Igfbp5 gene, or Tgβ2 gene) may be confirmed by RT-PCR or the like. The candidate substance is more accurately judged to be a hair growth-promoting substance or a hair growth-suppressing substance from the presence or absence of the formation of the hair shaft-like structure and the expression amount of the marker gene.

### EXAMPLES

Now, the present invention will be described by way of Examples, but the present invention is not limited to Examples to be described below.

### [Example 1] Manufacture of Plurality of Regenerated Mouse Hair Follicle Germs (Addition of Activated PRP)

1. Production of Microwell Plate

A schematic diagram of a production method for a microwell plate is illustrated in FIG. 5. Specifically, through the use of CAD software (V Carve Pro 6.5), the pattern of a microwell plate to be produced was designed on a computer. Subsequently, through the use of a cutting machine, an olefin-based substrate was cut according to the designed pattern to produce a concave mold having a pattern (step (I)). An epoxy resin (CRYSTAL RESIN: manufactured by Nissin Resin Co., Ltd.) was poured into the concave mold (step (II)), cured for 1 day (step (III)), and then released to form a convex mold having a pattern (step (IV)). Subsequently, the formed convex mold was fixed to the bottom surface of a 6 cm dish, and polydimethylsiloxane (PDMS) was poured into the dish (step (V)) and solidified (step (VI)). Subsequently, the solidified product was released to produce a microwell plate in which a regular pattern was formed in PDMS (step (VII)). The pattern design of the microwell plate was produced in accordance with the average hair follicle density of the hair of Japanese people. With regard to the size of the microwell plate, specifically, there was obtained a container measuring 1 cm high and 2 cm×2 cm square in which wells each having a diameter of about 1 mm and a height of 500 μm were arranged in the bottom surface of the container at a density of about 100 wells/cm².

2. Preparation of Platelet Rich Plasma (Activated PRP)

Next, platelet rich plasma (PRP) was prepared. Specifically, first, the abdomen of a pregnant mouse under inhalation anesthesia was cut open, and a 26G needle was inserted into the heart to collect about 1 mL of blood in a syringe (At this time, the syringe had been loaded in advance with 0.1 mL of an anticoagulant ACD-A solution). The composition of the anticoagulant ACD-A solution is 2.2 wt % Na citrate, 0.8 wt % citric acid dihydrate, and 2.2 wt % glucose. Subsequently, the collected blood was centrifuged at 1,560×g for 10 minutes to remove sedimented red blood cells to give a supernatant. Further, the resultant supernatant was centrifuged at 2,340×g for 10 minutes to collect PRP. The collected PRP and a 10 wt % calcium chloride solution were mixed at 7:1 to form a gel. The formed gel was centrifuged at 16,100×g for 10 minutes to collect an activated PRP (PRP-releasate; PRPr) solution.

3. Formation of Hair Follicle Germs

(1) Collection of Mesenchymal Cells and Epithelial Cells

Dorsal skin was collected from a C57BL/6 mouse embryo at embryonic day 18, and mesenchymal cells and epithelial cells were collected therefrom using a method obtained by partially modifying a method reported by Toyoshima et al. (Non Patent Literature 2). More specifically, the dorsal skin of a mouse embryo at embryonic day 18 in the uterus of a pregnant C57BL/6jjcl mouse was collected, and treated with Dispase (trademark) II (manufactured by Wako) at 4° C. under the shaking condition of 30 rpm for 1 hour to separate the epithelial layer and the mesenchymal layer. Then, the epithelial layer was treated with 100 U/mL collagenase (manufactured by Wako) for 2 hours and was further treated with trypsin for 10 minutes to isolate epithelial cells. Meanwhile, the mesenchymal layer was treated with 100 U/mL collagenase (manufactured by Wako) for 2 hours to isolate mesenchymal cells.

(2) Step of Forming Hair Follicle Germs

Subsequently, a mixed cell suspension of the collected epithelial cells and mesenchymal cells was poured into a poloxamer-treated microwell plate at 4×10³ cells/well for each kind of cell (total number of cells: 8×10³ cells/well), and the cells were cultured for 3 days. The composition of a medium used (hereinafter sometimes referred to as “PRPr+”) is as shown in Table 1 below. In addition, as a control, the cells were similarly cultured for 3 days using a medium free of the activated PRP (PRPr) solution and obtained by mixing a mesenchymal cell culture medium and an epithelial cell medium at 1:1 (hereinafter sometimes referred to as “PRPr−”). The medium was changed every day.

TABLE 1

Mixing ratio

| Compositional component | PRPr+ | PRPr− |
|---|---|---|
| Mesenchymal cell culture medium | 7.5 | 7.5 |
| (DMEM*¹ + 10% FBS*² + 1% P/S*³) | | |
| Epithelial cell culture medium | 7.5 | 7.5 |
| (HuMedia-KG2 medium (manufactured by | | |
| Kurabo Industries Ltd.)) | | |
| Activated PRP (PRPr) solution | 1 | — |

*¹DMEM: Dulbecco's Modified Eagle Medium
*²FBS: Fetal Bovine Serum
*³P/S: Penicillin/Streptomycin

(3) Observation Results

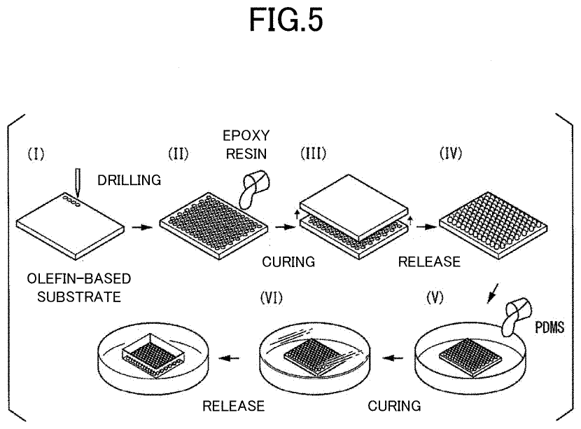

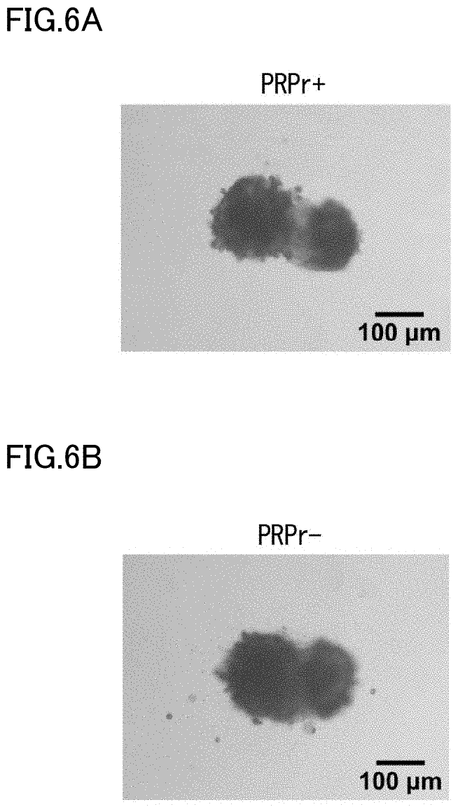

On the 3rd day from the start of culture, observation was performed using an inverted phase-contrast microscope (IX-71, MI-IBC, manufactured by Olympus). The results are shown in FIG. 6A (PRPr+) and FIG. 6B (PRPr−).

As shown in FIG. 6A and FIG. 6B, irrespective of the presence or absence of the addition of PRPr, the two kinds of cells inoculated formed one aggregate, and then the cells of the same kind aggregated with each other in the aggregate to form a hair follicle germ during the 3 days of culture.

(4) RT-PCRAnalysis for Versican Gene, Nexin Gene, Igfbp5 Gene, and Tgβ2 Gene

The hair follicle germs formed under the PRPr+ or PRPr− condition in (2) were evaluated for the expressions of hair growth-associated marker genes (Versican gene, Nexin gene, Igfbp5 gene, and Tgβ2 gene).

(4-1) RNA Extraction

First, the hair follicle germs formed under the PRPr+ or PRPr− condition were each collected in a 15 mL tube, and when each hair follicle germ was sedimented, the medium in the supernatant was removed so as to give 1 mL of a solution. Then, the resultant 1 mL of the solution containing each hair follicle germ was transferred to a 1.5 mL microtube.

Then, the microtube was centrifuged at 4° C. and 5,000 rpm for 3 minutes. This operation was performed in order to sediment each hair follicle germ to discard the remaining medium, and also to precool the inside of the centrifuge. Then, the supernatant (medium) was discarded, and then 350 μL of Buffer RLT was added, followed by thorough pipetting. Then, the solution after the pipetting was collected in a QIA Shredder spin column, and centrifuged at 4° C. and 10,000 rpm for 2 minutes. Then, an upper portion of the QIA Shredder spin column was discarded, 350 μL of 70% ethanol was added to the solution in the collection tube, and the resultant solution was transferred to an RNeasy spin column. Then, the column was centrifuged at 4° C. and 10,000 rpm for 15 seconds. Then, the filtrate in the collection tube was discarded, and 700 μL of Buffer RW1 was added, followed by centrifugation at 4° C. and 10,000 rpm for 15 seconds. Then, the filtrate in the collection tube was discarded, and 500 μL of Buffer RPE was added, followed by centrifugation at 4° C. and 10,000 rpm for 15 seconds. Then, the filtrate in the collection tube was discarded, and 500 μL of Buffer RPE was added, followed by centrifugation at 4° C. and 10,000 rpm for 2 minutes. Then, the column after the centrifugation was transferred to a fresh 2 mL collection tube, and centrifuged at 4° C. and 10,000 rpm for 1 minute. This was performed in order to remove the remaining Buffer RPE. Then, the column after the centrifugation was transferred to a 1 mL microtube, and 30 μL of RNase free water was added, followed by centrifugation at 4° C. and 10,000 rpm for 1 minute. Then, 30 μL of RNase free water was added again to the 1 mL microtube having the column after the centrifugation placed therein, followed by centrifugation at 4° C. and 10,000 rpm for 1 minute to afford an RNA solution.

(4-2) RT-PCR

Then, each resultant RNA solution was measured for its RNA concentration and diluted to 150 μg/mL. Then, the diluted RNA solution was incubated at 65° C. for 5 minutes, and then cooled on ice. Then, a solution having the composition shown in Table 2 below was added to the microtube, and the microtube was covered with a transparent film. In Table 2, RNA represents the RNA solution of the hair follicle germs formed under the PRPr+ or PRPr− condition.

TABLE 2

| Kind | Amount [μL] |
|---|---|
| Nuclear free water | 12 |
| 5 × RT Buffer | 4 |
| Primer mix | 1 |
| Enzyme mix | 1 |
| RNA | 2 |
| Total | 20 |

Then, the microtube was set in a thermal cycler, and it was confirmed that its lid was firmly closed. Then, a reverse transcription reaction was performed at 37° C. for 15 minutes and at 98° C. for 5 minutes to afford each of cDNA that was the reverse transcript of the RNA of the hair follicle germs formed under the PRPr+ condition, and cDNA that was the reverse transcript of the RNA of the hair follicle germs formed under the PRPr− condition. Then, a solution having the composition shown in Table 3 below was added to the microtube, and the microtube was covered with a transparent film. In Table 3, DNA represents each of the cDNA that was the reverse transcript of the RNA of the hair follicle germs formed under the PRPr+ condition, and the cDNA that was the reverse transcript of the RNA of the hair follicle germs formed under the PRPr− condition. Further, the base sequences of primers used in the PCR are shown in Table 4.

TABLE 3

| Kind | Amount [μL] |
|---|---|
| SYBR Green master mix | 10 |
| Forward Primer | 0.4 |
| Reverse Primer | 0.4 |
| Dye | 0.4 |
| Nuclear free water | 7.8 |
| DNA | 1 |
| Total | 20 |

TABLE 4

| | | SEQ | | SEQ |
|---|---|---|---|---|
| | | ID | | ID |
| | Forward primer | NO | Reverse primer | NO |
| Versican | 5′-GACGACTGTCTTGGTGG-3′ | 1 | 5′-ATATCCAAACAAGCCTG-3′ | 2 |
| Nexin | 5′-CCACGCAAAGCCAAGACGAC-3′ | 3 | 5′-gAAACCggCCTgCTCATCCT-3′ | 4 |
| Igfbp5 | 5′-ATGAGACAGGAATCCGAACA-3′ | 5 | 5′-TCAACGTTACTGCTGTCGAA-3′ | 6 |
| Tgfβ2 | 5′-TCCCGAATAAAAGCGAAGAG-3′ | 7 | 5′-AAGCTTCGGGATTTATGGTG-3′ | 8 |
| GAPDH | 5′-AGAACATCATCCCTGCATCC-3′ | 9 | 5′-TCCACCACCCTGTTGCTGTA-3′ | 10 |

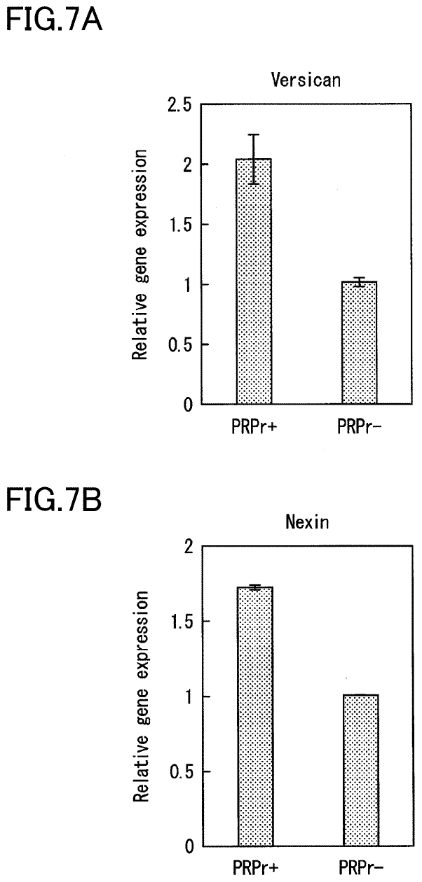

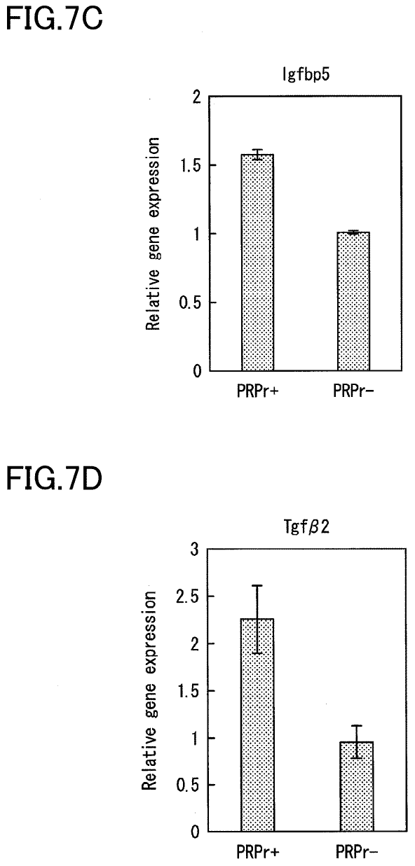

Then, the microtube was set in a thermal cycler, and it was confirmed that its lid was firmly closed. Then, PCR was performed by the following protocol: 95° C. for 4 minutes, (95° C. for 5 seconds and 60° C. for 60 seconds)×45 cycles, and 72° C. for 10 minutes. As a control, the expression amount of GAPDH was measured. The results are shown in FIG. 7A (Versican), FIG. 7B (Nexin), FIG. 7C (Igfbp5), and FIG. 7D (Tgβ2). In FIG. 7A to FIG. 7D, the expression amount of each gene under the PRPr+ condition when the expression amount of each gene under the PRPr− condition is defined as 1 is shown. As shown in FIG. 7A to FIG. 7D, compared to the hair follicle germs formed under the PRPr− condition, the hair follicle germs formed under the PRPr+ condition showed an increase in expression of each of the Versican, TGEβ2, Nexin, and Igfbp5 genes.

This suggested the possibility that PRPr promotes hair regeneration in the culture of hair follicle germs.

### [Test Example 1] Subcutaneous Transplantation Test Using Nude Mouse 1

(1) Subcutaneous Transplantation to Nude Mouse

The hair follicle germs formed under the PRPr+ condition obtained in Example 1 were intradermally transplanted to a nude mouse using a patch method. Animal care and animal experiments were performed in conformity with the guidelines of the animal experimental committee at Yokohama National University. As a control, the hair follicle germs formed under the PRPr− condition were also similarly transplanted.

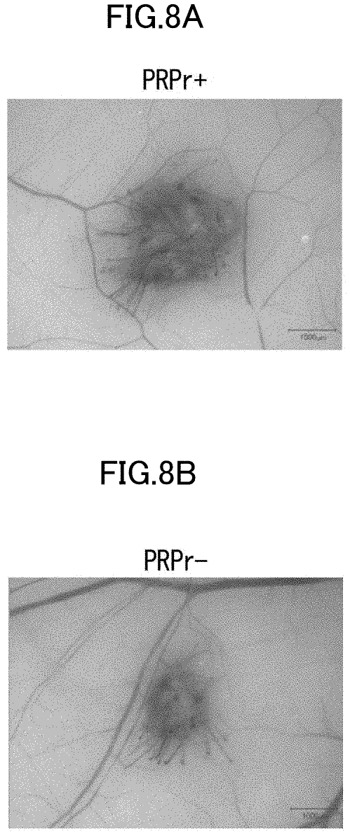

Specifically, first, a nude mouse was anesthetized by inhalation of isoflurane, and the dorsal part was disinfected with Isodine. Then, a V-lance micro-scalpel (manufactured by Alcon Japan Ltd.) was used to form incisions for transplantation ranging from the epidermal layer of the skin to a lower part of the dermal layer. Then, 30 hair follicle germs formed under the PRPr+ condition obtained in Example 1 per site, or 30 hair follicle germs formed under the PRPr− condition per site were inserted into a total of three incised sites for transplantation (90 germs in total). The states of regenerated hair at the transplantation portion of the nude mouse after 3 weeks from the transplantation are shown in FIG. 8A (PRPr+) and FIG. 8B (PRPr−).

As shown in FIG. 8A and FIG. 8B, a large number of black hairs were formed at the transplantation portion in which the hair follicle germs formed under the PRPr+ condition had been transplanted. In addition, it was observed that the number of regenerated hairs was larger compared to the transplantation portion in which the hair follicle germs formed under the PRPr− condition had been transplanted. In addition, at the transplantation portion in which the hair follicle germs formed under each of the PRPr+ and PRPr− conditions had been transplanted, the regenerated hairs were formed as morphologically normal hairs having a cuticle structure.

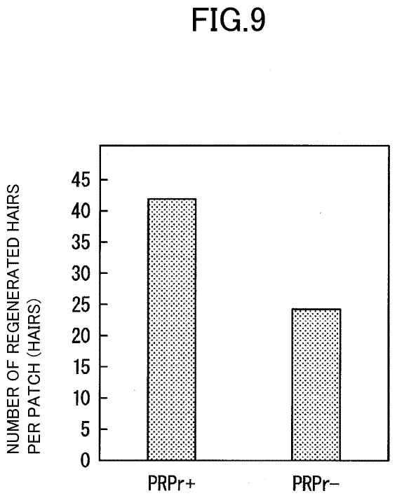

In addition, the skin at the transplantation portion was collected and immersed in a collagenase solution for 1 hour. After that, the regenerated hairs were loosened into separate hairs, and the number of regenerated hairs was counted. The results are shown in FIG. 9.

As shown in FIG. 9, the number of regenerated hairs for the hair follicle germs formed under the PRPr+ condition was about 42, which was about 1.75 times as large as that for the hair follicle germs formed under the PRPr− condition, which was about 24.

Thus, it was demonstrated that hair follicle germs cultured in a medium containing platelet rich plasma had a high hair follicle-inducing ability.

### [Example 2] Manufacture of Plurality of Regenerated Mouse Hair Follicle Germs (Addition of FGF)

Next, with attention focused on fibroblast growth factor 2 (FGF2), which is a hair regeneration factor abundantly contained in PRPr, an investigation was undertaken on an improvement in hair follicle-inducing ability by the addition of FGF2 to hair follicle germs. FGF2 is also used for HARG therapy, which is an alopecia treatment method. In addition, PRPr generally contains about 250 ng/mL of FGF2, and the FGF2 concentration in the medium used at the time of the formation of hair follicle germs in Example 1 was about 15 ng/mL. 1. Formation of Hair Follicle Germs

(1) Collection of Mesenchymal Cells and Epithelial Cells

Mesenchymal cells and epithelial cells were collected from a mouse embryo using the same method as in “3.(1)” of Example 1.

(2) Step of Forming Hair Follicle Germs

Subsequently, a mixed cell suspension of the collected epithelial cells and mesenchymal cells was poured into a 96-well microplate (manufactured by Sumitomo Bakelite Co., Ltd., Primesurface (trademark) 96 U Plate) at 1.5×10³ cells/well for each kind of cell (total number of cells: 3×10³ cells/well), and the cells were cultured. The composition of a medium used (hereinafter sometimes referred to as “FGF2+”) is as shown in Table 5 below. In addition, as a control, the cells were similarly cultured using a medium free of fibroblast growth factor 2 (FGF2) and obtained by mixing a mesenchymal cell culture medium and an epithelial cell medium at 1:1 (hereinafter sometimes referred to as “FGF2-”). The medium was changed every 2 to 3 days.

TABLE 5

Mixing ratio (content)

| Compositional component | FGF2+ | FGF2− |
|---|---|---|
| Mesenchymal cell culture medium | 1 | 1 |
| (DMEM*¹ + 10% FBS*² + 1% P/S*³) | | |
| Epithelial cell culture medium | 1 | 1 |
| (HuMedia-KG2 medium (manufactured | | |
| by Kurabo Industries Ltd.)) | | |
| FGF2*⁴ (manufactured by Ray | (10 ng/mL) | — |
| Biotech Inc. ) | | |

*¹DMEM: Dulbecco's Modified Eagle Medium
*²FBS: Fetal Bovine Serum
*³P/S: Penicillin/Streptomycin
*⁴FGF2: Fibroblast Growth Factor 2

(3) Observation Results

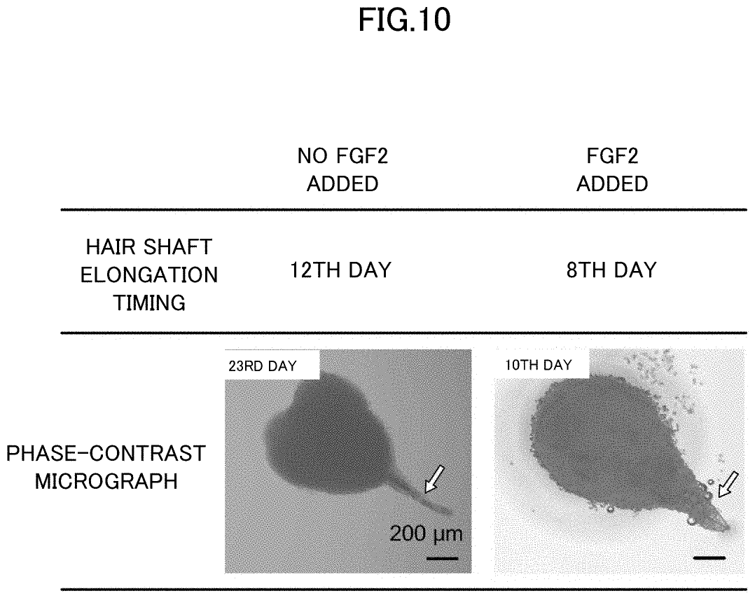

Observation was performed every day from the start of culture using an inverted phase-contrast microscope (IX-71, MI-IBC, manufactured by Olympus). The results are shown in FIG. 10. In FIG. 10, for the mesenchymal cells and epithelial cells cultured using the FGF2-free medium, a micrograph taken on the 23rd day of culture is shown. Meanwhile, for the mesenchymal cells and epithelial cells cultured using the FGF2-containing medium, a micrograph taken on the 10th day of culture is shown.

On the 3rd day of culture, irrespective of the presence or absence of the addition of FGF2, the two kinds of cells inoculated formed one aggregate, and then the cells of the same kind aggregated with each other in the aggregate to form a hair follicle germ during the 3 days of culture (not shown).

After that, in the hair follicle germs formed under the FGF2+ condition, the formation of a hair shaft-like structure was observed on the 8th day of culture. Meanwhile, in the hair follicle germs formed under the FGF2− condition, the formation of a similar hair shaft-like structure was observed on the 12th day of culture.

Thus, it is conceivable that, in the in-vitro test system, the addition of FGF2 promoted hair regeneration with the hair follicle germs, and hence the hair shaft-like structure was observed earlier. The formation of the hair shaft-like structure with the hair follicle germs in the in-vitro test system is an extremely interesting phenomenon. Therefore, in view of the fact that the FGF serving as a hair growth factor promoted the formation of the hair shaft-like structure, the following applications are expected: an evaluation tool for a hair growth agent or the like, and screening for a candidate compound having a hair growth-promoting action or a hair growth-suppressing action.

(4) RT-PCR Analysis for Versican Gene and Tgβ2 Gene

The hair follicle germs formed under the FGF2+ or FGF2-condition in (2) (10th day of culture) were evaluated for the expressions of hair growth-associated marker genes (Versican gene and Tgβ2 gene).

(4-1) RNA Extraction

An RNA solution was prepared using the same method as in (4-1) of Example 1.

(4-2) RT-PCR

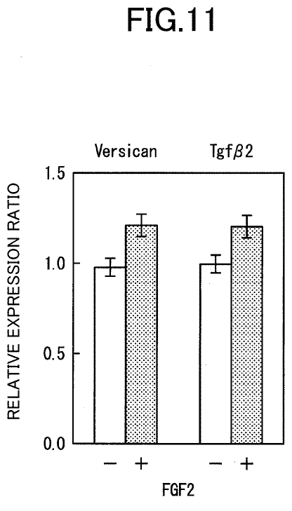

The RNA solution obtained in (4-1) was subjected to RT-PCR using the same method as in (4-2) of Example 1 to afford each of cDNA that was the reverse transcript of the RNA of the hair follicle germs formed under the FGF2+ condition, and the cDNA that was the reverse transcript of the RNA of the hair follicle germs formed under the FGF2− condition. A solution having the composition shown in Table 3 above was added to the resultant cDNA, and PCR was further performed using the primers for the Versican gene, the Tgβ2 gene, and the GAPDH gene shown in Table 4 above. The results are shown in FIG. 11.

As shown in FIG. 11, compared to the hair follicle germs formed under the FGF2− condition, the hair follicle germs formed under the FGF2+ condition showed an increase in expression of each of the Versican and Igfbp5 genes.

This suggested the possibility that FGF2 promotes hair regeneration in the culture of hair follicle germs.

### [Reference Example 1] Manufacture of Plurality of Regenerated Mouse Hair Follicle Germs (Addition of Non-Activated PRP)

1. Production of Microwell Plate

A microwell plate was produced using the same method as in “1.” of Example 1.

2. Preparation of Platelet Rich Plasma (Non-Activated PRP)

Next, platelet rich plasma (non-activated PRP) (hereinafter sometimes referred to simply as “PRP”) was prepared. Specifically, first, the abdomen of a pregnant mouse under inhalation anesthesia was cut open, and a 26G needle was inserted into the heart to collect about 1 mL of blood in a syringe (At this time, the syringe had been loaded in advance with 0.1 mL of an anticoagulant ACD-A solution). The composition of the anticoagulant ACD-A solution is 2.2 wt % Na citrate, 0.8 wt % citric acid dihydrate, and 2.2 wt % glucose. Subsequently, the collected blood was centrifuged at 1,560×g for 10 minutes to remove sedimented red blood cells to give a supernatant. Further, the resultant supernatant was centrifuged at 2,340×g for 10 minutes to collect PRP (non-activated PRP).

3. Formation of Hair Follicle Germs

(1) Collection of Mesenchymal Cells and Epithelial Cells

Mesenchymal cells and epithelial cells were collected from a mouse embryo using the same method as in “3.(1)” of Example 1.

(2) Step of Forming Hair Follicle Germs

Subsequently, a mixed cell suspension of the collected epithelial cells and mesenchymal cells was poured into a poloxamer-treated microwell plate at 4×10³ cells/well for each kind of cell (total number of cells: 8×10³ cells/well), and the cells were cultured for 3 days. The composition of a medium used is as shown in Table 6 below. In addition, as a control, the cells were similarly cultured for 3 days using a medium free of the platelet rich plasma solution and obtained by mixing a mesenchymal cell culture medium and an epithelial cell medium at 1:1 (hereinafter sometimes referred to as “PRP−” or “PRP− (0%)”). The medium was changed every day.

TABLE 6

| | Mixing ratio | | | |
|---|---|---|---|---|
| | PRP+ | PRP+ | PRP+ | PRP− |
| Compositional component | (20%) | (10%) | (5%) | (0%) |
| Mesenchymal cell culture medium | 8 | 9 | 9.5 | 10 |
| (DMEM*¹ + 10% FBS*² + 1% P/S*³) | | | | |
| Epithelial cell culture medium | 8 | 9 | 9.5 | 10 |
| (HuMedia-KG2 medium (manufactured | | | | |
| by Kurabo Industries Ltd.)) | | | | |
| Non-activated PRP | 4 | 2 | 1 | 0 |

*¹DMEM: Dulbecco's Modified Eagle Medium
*²FBS: Fetal Bovine Serum
*³P/S: Penicillin/Streptomycin

(3) Observation Results

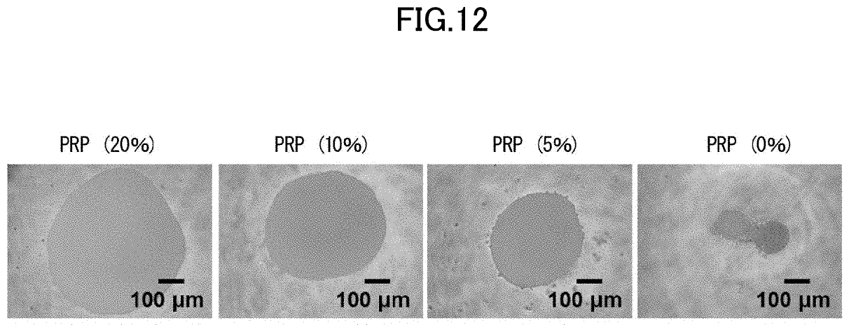

On the 3rd day from the start of culture, observation was performed using an inverted phase-contrast microscope (IX-71, MI-IBC, manufactured by Olympus). The results are shown in FIG. 12.

As shown in FIG. 12, under PRP+, at each of the concentrations, the formation of hair follicle germs was inhibited and one spherical tissue body was formed. The size of the spherical tissue body depended on the addition concentration of PRP, and hence it was presumed that platelets and the like formed the tissue body formed of the epithelial cells and the mesenchymal cells.

(4) RT-PCR Analysis for Versican Gene

The tissue body formed under the PRP+(5%) condition or the hair follicle germs formed under the PRP− condition in (2) (3rd day of culture) were evaluated for the expression of a hair growth-associated marker gene (Versican gene).

(4-1) RNA Extraction

An RNA solution was prepared using the same method as in (4-1) of Example 1.

(4-2) RT-PCR

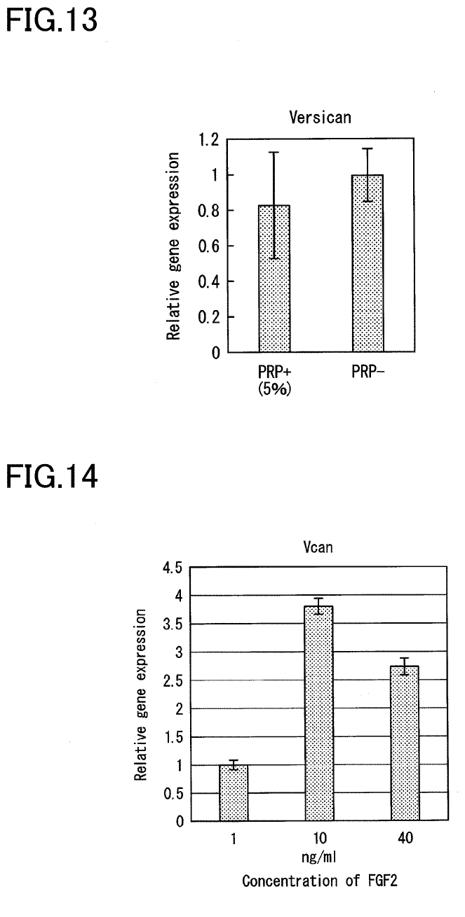

The RNA solution obtained in (4-1) was subjected to RT-PCR using the same method as in (4-2) of Example 1 to afford each of cDNA that was the reverse transcript of the RNA of the tissue body formed under the PRP+(5%) condition, and the cDNA that was the reverse transcript of the RNA of the hair follicle germs formed under the PRP− condition. A solution having the composition shown in Table 3 above was added to the resultant cDNA, and PCR was further performed using the primers for the Versican gene and the GAPDH gene shown in Table 4 above. The results are shown in FIG. 13.

As shown in FIG. 13, as a result of comparing the expression of the Versican gene in the tissue body formed under the PRP+(5%) condition, and that in the hair follicle germs formed under the PRP− condition, it was found that the expression of the Versican gene was hardly improved in the tissue body formed under the PRP+(5%) condition. This was presumably because the platelets contained in the PRP were not activated.

In Example 1, activated PRP was obtained through contact with a calcium solution, and hence it was revealed that the use of the activated PRP was able to improve the hair regeneration ability of hair follicle germs.

### [Example 3] Manufacture of Plurality of Regenerated Mouse Hair Follicle Germs (Investigation on Addition Amount of FGF 1)

1. Production of Microwell Plate

A microwell plate was produced using the same method as in “1.” of Example 1.

2. Preparation of Platelet Rich Plasma (Activated PRP)

A platelet rich plasma (activated PRP (PRPr)) solution was prepared using the same method as in “2.” of Example 1.

3. Formation of Hair Follicle Germs

(1) Collection of Mesenchymal Cells and Epithelial Cells

Mesenchymal cells and epithelial cells were collected from a mouse embryo using the same method as in “3.(1)” of Example 1.

(2) Step of Forming Hair Follicle Germs

Subsequently, a mixed cell suspension of the collected epithelial cells and mesenchymal cells was poured into a poloxamer-treatedmicrowell plate at 4×10³ cells/well for each kind of cell (total number of cells: 8×10³ cells/well), and the cells were cultured for 3 days. The composition of a medium used is as shown in Table 7 below. In addition, as controls, the cells were similarly cultured for 3 days using a medium free of FGF2 and obtained by mixing a mesenchymal cell culture medium and an epithelial cell medium at 1:1 (hereinafter sometimes referred to as “FGF2-/PRPr-”), and a medium free of FGF2 and obtained by mixing the activated PRP solution, the mesenchymal cell culture medium, and the epithelial cell medium at a mixing ratio shown in Table 7 (hereinafter sometimes referred to as “FGF2-/PRPr+”). The medium was changed every day.

TABLE 7

| | Mixing ratio (content) | | | | |
|---|---|---|---|---|---|
| Compositional | | | | FGF2− | |
| component | FGF2+ | | | PRPr+ | PRPr− |
| Mesenchymal cell | 1 | 1 | 1 | 9.5 | 1 |
| culture medium | | | | | |
| (DMEM*¹ + 10% | | | | | |
| FBS*² + 1% P/S*³) | | | | | |
| Epithelial cell | 1 | 1 | 1 | 9.5 | 1 |
| culture medium | | | | | |
| (HuMedia-KG2 medium | | | | | |
| (manufactured by | | | | | |
| Kurabo Industries Ltd.)) | | | | | |
| Activated PRP (PRPr) | — | — | — | 1 | — |
| solution | | | | | |
| FGF2*⁴ | (1 | (10 | (40 | — | — |
| (manufactured by Ray | ng/mL) | ng/mL) | ng/mL) | | |
| Biotech Inc.) | | | | | |

*¹DMEM: Dulbecco's Modified Eagle Medium
*²FBS: Fetal Bovine Serum
*³P/S: Penicillin/Streptomycin
*⁴FGF2: Fibroblast Growth Factor 2

(3) RT-PCR Analysis for Versican Gene

The hair follicle germs formed under each condition in (2) (3rd day of culture) were evaluated for the expression of a hair growth-associated marker gene (Versican gene).

(3-1) RNA Extraction

An RNA solution was prepared using the same method as in (4-1) of Example 1.

(3-2) RT-PCR

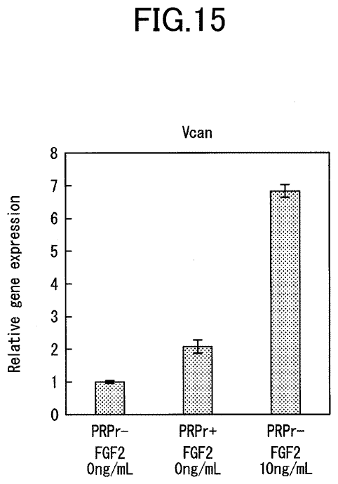

The RNA solution obtained in (3-1) was subjected to RT-PCR using the same method as in (4-2) of Example 1 to afford each cDNA that was the reverse transcript of the RNA of the hair follicle germs formed under each condition. A solution having the composition shown in Table 3 above was added to the resultant cDNA, and PCR was further performed using the primers for the Versican gene and the GAPDH gene shown in Table 4 above. The results are shown in FIG. 14 (only FGF2+) and FIG. 15 (FGF2−/PRPr−, FGF2−/PRPr+, and FGF2+(10 ng/mL)). In FIG. 14 and FIG. 15, “Vcan” is an abbreviation for Versican.

As shown in FIG. 14, the expression of the Versican gene showed the highest value in the hair follicle germs cultured under the condition of an FGF2 content of 10 ng/mL.

In addition, as shown in FIG. 15, the hair follicle germs cultured under the FGF2+(10 ng/mL) condition had a higher expression of the Versican gene than the hair follicle germs cultured under the FGF2-/PRPr+ condition. Specifically, the expression amount of the former was 3 or more times as high as that of the latter.

Thus, it was suggested that, of the components contained in PRPr, FGF2 highly contributed to the promotion of hair follicle regeneration.

### [Example 4] Manufacture of Plurality of Regenerated Hair Follicle Germs Each Having Capillary Structure

1. Production of Microwell Plate

A microwell plate was produced using the same method as in “1.” of Example 1.

2. Preparation of Platelet Rich Plasma (Activated PRP)

A platelet rich plasma (activated PRP (PRPr)) solution was prepared using the same method as in “2.” of Example 1.

3. Formation of Hair Follicle Germs

(1) Collection of Mesenchymal Cells and Epithelial Cells

Mesenchymal cells and epithelial cells were collected from a mouse embryo using the same method as in “3.(1)” of Example 1.

(2) Preparation of Vascular Endothelial Cells

Human umbilical vein endothelial cells transfected with RFP gene (RFP-HUVEC) (hereinafter sometimes referred to simply as “vascular endothelial cells”) had been cultured to confluence in advance.

(3) Step of Forming Hair Follicle Germs

Subsequently, a mixed cell suspension of the epithelial cells, the mesenchymal cells, and the vascular endothelial cells was poured into a poloxamer-treated microwell plate at 4×10³ cells/well for each of the epithelial cells and the mesenchymal cells, and at 1×10³ cells/well for the vascular endothelial cells (total number of cells: 9×10³ cells/well), and the cells were cultured for 3 days. The composition of a medium used is as shown in Table 8 below. In addition, as controls, the cells were similarly cultured for 3 days using a medium free of FGF2 and obtained by mixing a mesenchymal cell culture medium and an epithelial cell medium at 1:1 (hereinafter sometimes referred to as “FGF2−/PRPr−”), and a medium free of FGF2 and obtained by mixing the activated PRP solution, the mesenchymal cell culture medium, and the epithelial cell medium at a mixing ratio shown in Table 8 (hereinafter sometimes referred to as “FGF2−/PRPr+”). The medium was changed every day.

TABLE 8

| | Mixing ratio (content) | | | |
|---|---|---|---|---|
| Compositional | | | FGF2− | |
| component | FGF2+ | | PRPr+ | PRPr− |
| Mesenchymal cell | 1 | 1 | 9.5 | 1 |
| culture medium | | | | |
| (DMEM*¹ + 10% | | | | |
| FBS*² + 1% P/S*³) | | | | |
| Epithelial cell | 1 | 1 | 9.5 | 1 |
| culture medium | | | | |
| (HuMedia-KG2 medium | | | | |
| (manufactured by | | | | |
| Kurabo Industries Ltd.)) | | | | |
| Activated PRP (PRPr) | — | — | 1 | — |
| solution | | | | |
| FGF2*⁴ (manufactured | (10 ng/mL) | (40 ng/mL) | — | — |
| by Ray Biotech Inc.) | | | | |

*¹DMEM: Dulbecco's Modified Eagle Medium
*²FBS: Fetal Bovine Serum
*³P/S: Penicillin/Streptomycin
*⁴FGF2: Fibroblast Growth Factor 2

(4) Observation Results

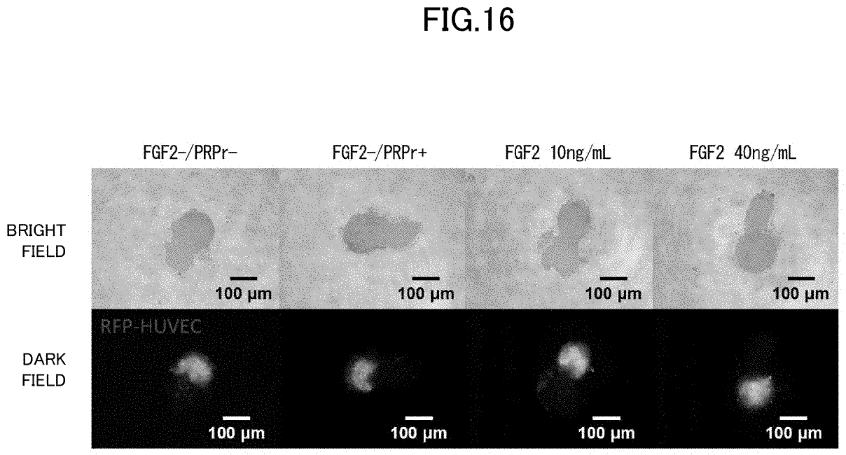

On the 3rd day from the start of culture, observation was performed using an inverted fluorescence microscope (DP-71, manufactured by Olympus). The results are shown in FIG. 16.

As shown in FIG. 16, irrespective of the presence or absence of the addition of FGF2 and PRPr, three kinds of inoculated cells formed one aggregate. Further, the cells of the same kind aggregated with each other in the aggregate to form a hair follicle germ having a vascular network during the 3 days of culture (see upper images of FIG. 16). The vascular network was formed only in the aggregate of the mesenchymal cells in the hair follicle germ (see lower images of FIG. 16).

(5) RT-PCR Analysis for Versican Gene

The hair follicle germs formed under each condition in (3) (3rd day of culture) were evaluated for the expression of a hair growth-associated marker gene (Versican gene).

(5-1) RNA Extraction

An RNA solution was prepared using the same method as in (4-1) of Example 1.

(5-2) RT-PCR

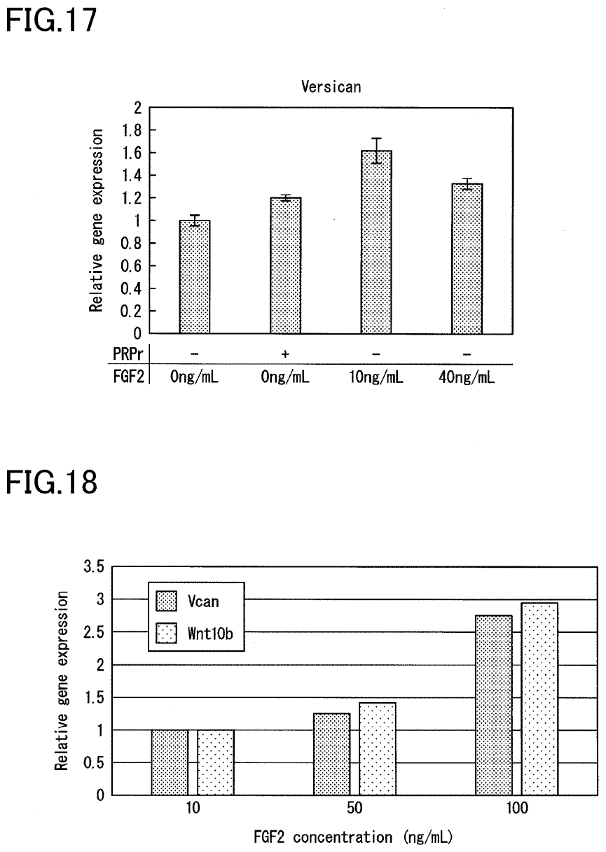

The RNA solution obtained in (5-1) was subjected to RT-PCR using the same method as in (4-2) of Example 1 to afford each cDNA that was the reverse transcript of the RNA of the hair follicle germs formed each condition. A solution having the composition shown in Table 3 above was added to the resultant cDNA, and PCR was further performed using the primers for the Versican gene and the GAPDH gene shown in Table 4 above. The results are shown in FIG. 17.

As shown in FIG. 17, the hair follicle germs cultured under the FGF2−/PRPr+, and FGF2+(10 ng/mL and 40 ng/mL) conditions had high expression amounts of the Versican gene compared to the hair follicle germs cultured under the FGF2-/PRPr− condition. In particular, the hair follicle germs cultured under the FGF2+(10 ng/mL) condition had the highest expression amount of the Versican gene.

### [Example 5] Manufacture of Plurality of Regenerated Mouse Hair Follicle Germs (Investigation on Addition Amount of FGF 2)

There is a report that cells collected from a mouse embryo have a high hair regeneration ability, and the ability is reduced as the mouse grows into an adult. In view of this, an investigation was undertaken on the addition amount of an FGF in the case of manufacturing hair follicle germs using cells collected from an adult mouse.

1. Production of Microwell Plate

A microwell plate was produced using the same method as in “1.” of Example 1.

2. Formation of Hair Follicle Germs

(1) Collection of Mesenchymal Cells and Epithelial Cells

Mesenchymal cells and epithelial cells were collected from the hair follicles of the whiskers of an adult mouse by using a method obtained by partially modifying a method reported by Toyoshima et al. (Non Patent Literature 2).

(2) Step of Forming Hair Follicle Germs

Subsequently, a mixed cell suspension of the collected epithelial cells and mesenchymal cells was poured into a poloxamer-treated microwell plate at 1×10⁴ cells/well for each kind of cell (total number of cells: 2×10⁴ cells/well), and the cells were cultured for 3 days. The composition of a medium used is as shown in Table 9 below. In addition, as a control, the cells were similarly cultured for 3 days using a medium free of FGF2 and obtained by mixing a mesenchymal cell culture medium and an epithelial cell medium at 1:1 (hereinafter sometimes referred to as “FGF2-”). The medium was changed every day.

TABLE 9

| | Mixing ratio (content) | | | | |
|---|---|---|---|---|---|
| Compositional component | FGF2+ | | | | FGF− |
| Mesenchymal cell | 1 | 1 | 1 | 1 | 1 |
| culture medium | | | | | |
| (DMEM*¹ + 10% | | | | | |
| FBS*² + 1% P/S*³) | | | | | |
| Epithelial cell | 1 | 1 | 1 | 1 | 1 |
| culture medium (HuMedia- | | | | | |
| KG2 medium (manufactured | | | | | |
| by Kurabo Industries Ltd.)) | | | | | |
| FGF2*⁴ (manufactured by | (1) | (10) | (50) | (100) | (0) |
| Ray Biotech Inc.) (ng/mL) | | | | | |

*¹DMEM: Dulbecco's Modified Eagle Medium
*²FBS: Fetal Bovine Serum
*³P/S: Penicillin/Streptomycin
*⁴FGF2: Fibroblast Growth Factor 2

(3) RT-PCR Analysis for Versican Gene and Wnt10b Gene

The hair follicle germs formed under each condition in (2) (3rd day of culture) were evaluated for the expressions of a marker gene for mesenchymal cells serving as an indicator for hair regeneration efficiency (Versican gene), and a marker gene for epithelial cells serving as an indicator for hair regeneration efficiency (Wnt10b gene).

(3-1) RNA Extraction

An RNA solution was prepared using the same method as in (4-1) of Example 1.

(3-2) RT-PCR

The RNA solution obtained in (3-1) was subjected to RT-PCR using the same method as in (4-2) of Example 1 to afford each cDNA that was the reverse transcript of the RNA of the hair follicle germs formed under each condition. A solution having the composition shown in Table 3 above was added to the resultant cDNA, and PCR was further performed using the primers for the Versican gene shown in Table 4 above, and primers for the Wnt10b gene shown in Table 10 below. As typical results, the relative expression amounts of each gene in the hair follicle germs cultured under the FGF2 conditions of 10 ng/mL, 50 ng/mL, and 100 ng/mL are shown in FIG. 18. In FIG. 18, “Vcan” is an abbreviation for Versican.

TABLE 10

| | | SEQ | | SEQ |
|---|---|---|---|---|
| | | ID | | ID |
| | Forward primer | NO | Reverse primer | NO |
| Wnt10b | 5′-CCAAGAGCCGGGCCCGAGTGA-3′ | 11 | 5′-AAGGGCGGAGGCCGAGACCG-3′ | 12 |

The following tendency was shown from FIG. 18: as the FGF2 content increased, the expressions of the Versican gene and the Wnt10b gene in the hair follicle germs increased.

In addition, as shown in FIG. 18, the hair follicle germs cultured under the FGF2+(100 ng/mL) condition had higher expressions of the Versican gene and the Wnt10b gene than the hair follicle germs cultured under the FGF2+(10 ng/mL) condition. Specifically, the expression amounts of the former were 3 or more times as high as those of the latter.

Thus, it was suggested that, in the case of using cells collected from an adult, it was effective to add a larger amount of FGF2.

### [Test Example 2] Subcutaneous Transplantation Test Using Nude Mouse 2

(1) Subcutaneous Transplantation to Nude Mouse

Of the hair follicle germs obtained in Example 5, the hair follicle germs formed under the FGF2+(10 ng/mL and 100 ng/mL) conditions were each intradermally transplanted to a nude mouse using the patch method in the same manner as in Test Example 1. As a control, the hair follicle germs formed under the FGF2− (0 ng/mL) condition were also similarly transplanted.

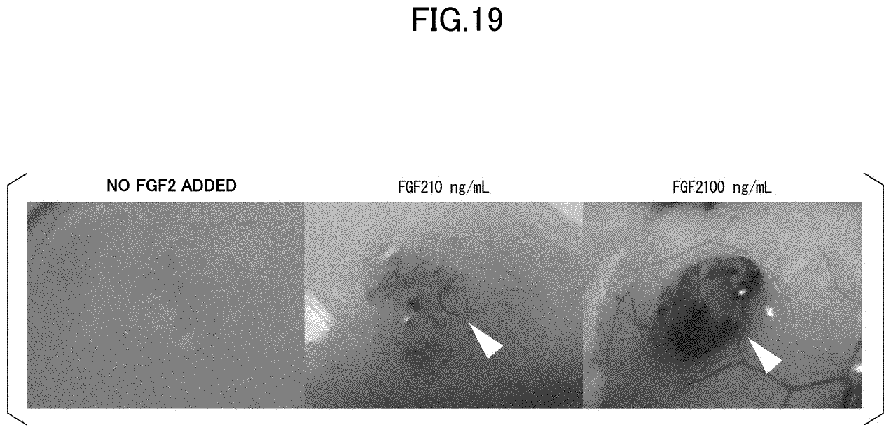

The states of regenerated hair at the transplantation portion of the nude mouse after 3 weeks from the transplantation are shown in FIG. 19.

As shown in FIG. 19, hair growth was not confirmed at the transplantation portion in which the hair follicle germs formed under the FGF2− (0 ng/mL) condition had been transplanted, whereas a large number of black hairs were formed in the case of the hair follicle germs formed under the FGF2+(10 ng/mL and 100 ng/mL) conditions. In addition, as the addition concentration of FGF2 increased, the number of regenerated hairs increased.

In addition, though not shown, hair regeneration was confirmed as a result of intradermal transplantation of hair follicle germs formed under an FGF2+(200 ng/mL) condition to a nude mouse using the same method as in Example 5.

### [Example 6] Manufacture of Plurality of Regenerated Mouse Hair Follicle Germs (Investigation on Effective Component in PRPr)

It is known that PRPr contains various cytokines other than FGFs. In view of this, an investigation was performed on the expression amounts of marker genes in the case of manufacturing hair follicle germs using a medium supplemented with PDGF or VEFG in place of FGF.

1. Production of Microwell Plate

A microwell plate was produced using the same method as in “1.” of Example 1.

2. Formation of Hair Follicle Germs

(1) Collection of Mesenchymal Cells and Epithelial Cells

Mesenchymal cells and epithelial cells were collected from a mouse embryo using the same method as in “3.(1)” of Example 1.

(2) Step of Forming Hair Follicle Germs

Subsequently, a mixed cell suspension of the collected epithelial cells and mesenchymal cells was poured into a poloxamer-treated microwell plate at 4×10³ cells/well for each kind of cell (total number of cells: 8×10³ cells/well), and the cells were cultured for 3 days. With regard to the composition of a medium used, a medium obtained by mixing a mesenchymal cell culture medium (DMEM+10% FBS+1% P/S) and an epithelial cell culture medium (HuMedia-KG2 medium (manufactured by Kurabo Industries Ltd.)) at 1:1, the medium being supplemented with FGF, PDGF, or VEGF each at 0 ng/mL, 1 ng/mL, 10 ng/mL, or 100 ng/mL, was used. The medium was changed every day.

(3) RT-PCR Analysis for Versican Gene and Wnt10b Gene

The hair follicle germs formed under each condition in (2) (3rd day of culture) were evaluated for the expressions of a marker gene for mesenchymal cells serving as an indicator for hair regeneration efficiency (Versican gene), and a marker gene for epithelial cells serving as an indicator for hair regeneration efficiency (Wnt10b gene).

(3-1) RNA Extraction

An RNA solution was prepared using the same method as in (4-1) of Example 1.

(3-2) RT-PCR

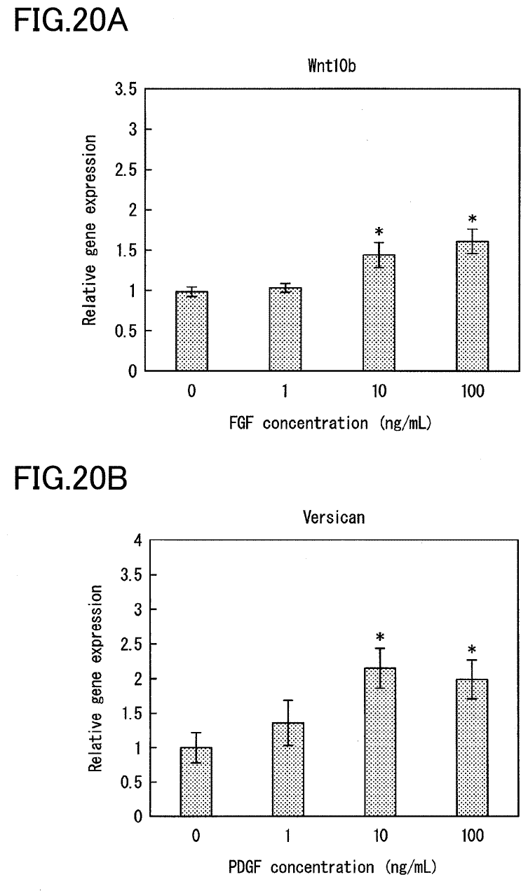

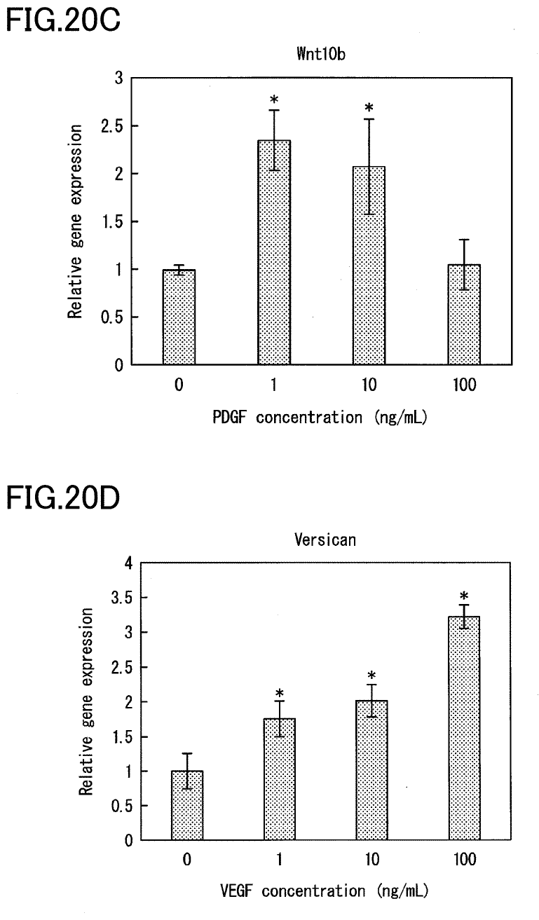

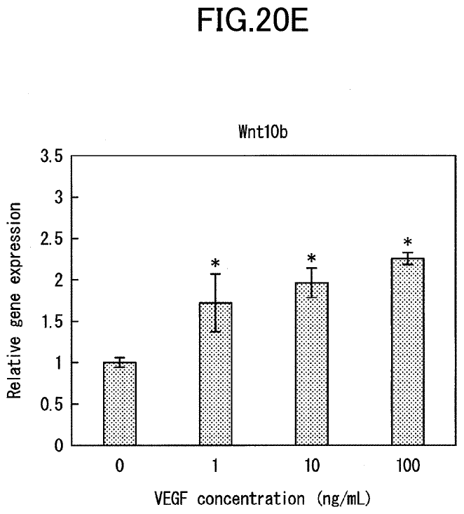

The RNA solution obtained in (3-1) was subjected to RT-PCR using the same method as in (4-2) of Example 1 to afford each cDNA that was the reverse transcript of the RNA of the hair follicle germs formed under each condition. A solution having the composition shown in Table 3 above was added to the resultant cDNA, and PCR was further performed using the primers for the Versican gene shown in Table 4 above, and the primers for the Wnt10b gene shown in Table 10 above. The results are shown in FIG. 20A to FIG. 20E.

It was revealed from FIG. 20A to FIG. 20E that each of FGF2, PDGF, and VEGF, components contained in PRPr, contributed to the improvement of the expressions of the Versican gene and the Wnt10b gene associated with hair regeneration.

### [Example 7] Manufacture of Plurality of Regenerated Mouse Hair Follicle Germs (Using Human Hair Papilla Cells)

1. Production of Microwell Plate

A microwell plate was produced using the same method as in “1.” of Example 1.

2. Formation of Hair Follicle Germs

(1) Preparation of Mesenchymal Cells and Collection of Epithelial Cells

Epithelial cells were collected from a mouse embryo using the same method as in “3.(1)” of Example 1. Human hair papilla cells (manufactured by PromoCell GmbH) were used as mesenchymal cells.

(2) Step of Forming Hair Follicle Germs

Subsequently, a mixed cell suspension of the epithelial cells and mesenchymal cells was poured into a poloxamer-treated microwell plate at 4×10³ cells/well for each kind of cell (total number of cells: 8×10³ cells/well), and the cells were cultured for 3 days. With regard to the composition of a medium used, a medium obtained by mixing a human hair papilla cell growth medium (Follicle Dermal Papilla Cell Growth Medium; DPCGM) (manufactured by PromoCell GmbH) and an epithelial cell medium (HuMedia-KG2 medium (manufactured by Kurabo Industries Ltd.)) at 1:1, the medium being supplemented with FGF at 10 ng/mL, was used. The medium was changed every day. After 3 days from the start of culture, it was confirmed that hair follicle germs had been formed.

### [Test Example 3] Subcutaneous Transplantation Test Using Nude Mouse 3

(1) Subcutaneous Transplantation to Nude Mouse

The hair follicle germs obtained in Example 7 were intradermally transplanted to a nude mouse using the patch method in the same manner as in Test Example 1. The state of regenerated hair at the transplantation portion of the nude mouse on the 18th day from transplantation is shown in FIG. 21.

As shown in FIG. 21, hair regeneration was observed at the transplantation portion transplanted with the hair follicle germs formed using human hair papilla cells as mesenchymal cells. In addition, it was confirmed that the hair cycle was maintained over at least half a year from the start of transplantation (not shown).

### [Example 8] Manufacture of Plurality of Regenerated Mouse Hair Follicle Germs (Using Human Cell System)

1. Production of Microwell Plate

A microwell plate was produced using the same method as in “1.” of Example 1.

2. Formation of Hair Follicle Germs

(1) Preparation of Mesenchymal Cells and Epithelial Cells

Human hair papilla cells (manufactured by PromoCell GmbH) were used as mesenchymal cells. In addition, human epidermal keratinocytes (manufactured by CELLnTEC) were used as epithelial cells.

(2) Step of Forming Hair Follicle Germs

Subsequently, a mixed cell suspension of the epithelial cells and mesenchymal cells was poured into a poloxamer-treated microwell plate at 1×10⁴ cells/well for each kind of cell (total number of cells: 2×10⁴ cells/well), and the cells were cultured for 3 days. With regard to the composition of a medium used, a medium obtained by mixing DPCGM (manufactured by PromoCell GmbH) and an epithelial cell medium (HuMedia-KG2 medium (manufactured by Kurabo Industries Ltd.)) at 1:1, the medium being supplemented with FGF at 10 ng/mL, was used. The medium was changed every day. After 3 days from the start of culture, it was confirmed that hair follicle germs had been formed.

### [Test Example 4] Subcutaneous Transplantation Test Using Nude Mouse 4

(1) Subcutaneous Transplantation to Nude Mouse

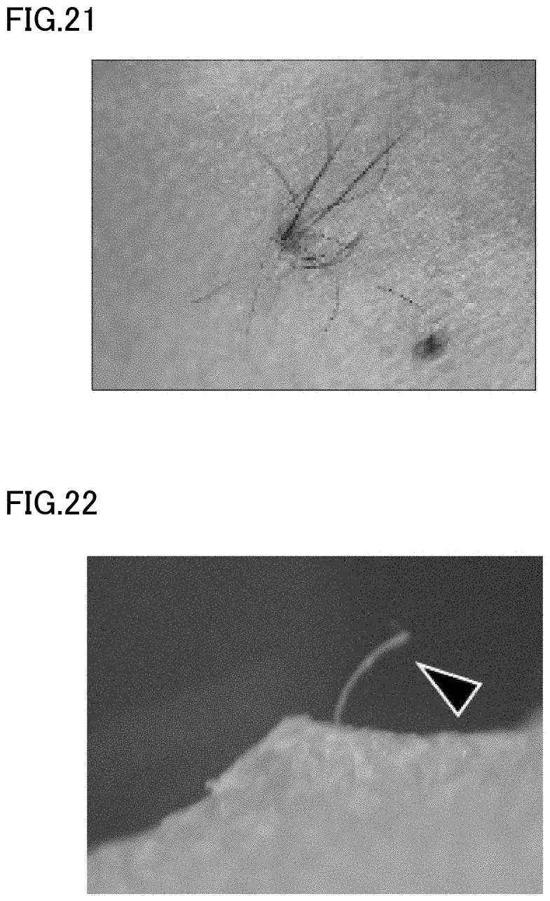

The hair follicle germs obtained in Example 8 were intradermally transplanted to a nude mouse using the patch method in the same manner as in Test Example 1. The state of regenerated hair at the transplantation portion of the nude mouse on the 14th day from transplantation is shown in FIG. 22.

As shown in FIG. 22, hair regeneration was observed at the transplantation portion transplanted with the hair follicle germs formed using human-derived cells as mesenchymal cells and epithelial cells. Owing to no inclusion of pigment cells (melanocytes), white hairs were regenerated.

### [Example 9] Manufacture of Plurality of RegeneratedMouse Hair Follicle Germs (Using Hair Papilla Cells Derived from Human Alopecia Patient)

1. Production of Microwell Plate

A microwell plate was produced using the same method as in “1.” of Example 1.

2. Formation of Hair Follicle Germs

(1) Preparation of Mesenchymal Cells and Epithelial Cells

Epithelial cells were collected from a mouse embryo using the same method as in “3. (1)” of Example 1. With the consent of a human alopecia patient, a normal scalp piece of the human alopecia patient was collected, and hair papilla cells were separated from the normal scalp piece and used as mesenchymal cells.

(2) Step of Forming Hair Follicle Germs

Subsequently, a mixed cell suspension of the collected epithelial cells and mesenchymal cells was poured into a poloxamer-treated microwell plate at 1×10⁴ cells/well for each kind of cell (total number of cells: 2×10⁴ cells/well), and the cells were cultured for 3 days. With regard to the composition of a medium used, a medium obtained by mixing DPCGM (manufactured by PromoCell GmbH) and an epithelial cell medium (HuMedia-KG2 medium (manufactured by Kurabo Industries Ltd.)) at 1:1, the medium being supplemented with FGF at 100 ng/mL, was used. The medium was changed every day. After 3 days from the start of culture, it was confirmed that hair follicle germs had been formed.

### [Test Example 5] Subcutaneous Transplantation Test Using Nude Mouse 5

(1) Subcutaneous Transplantation to Nude Mouse

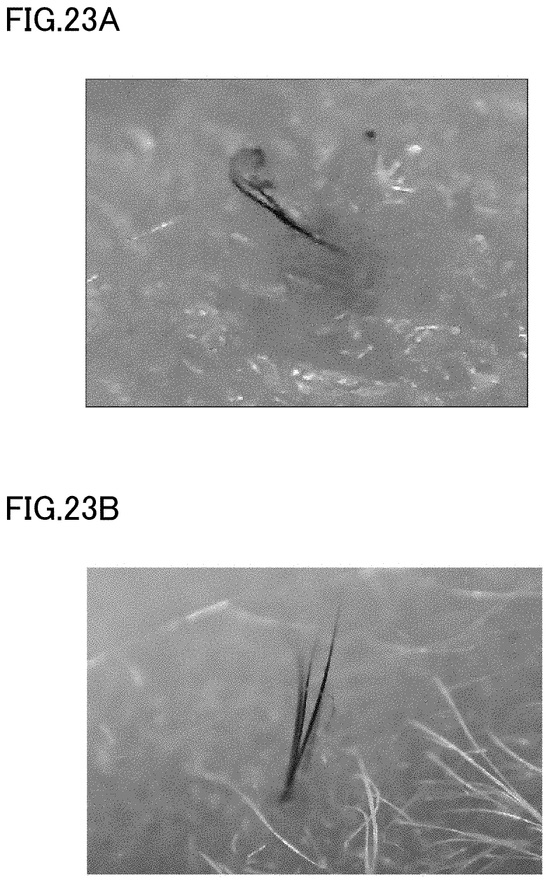

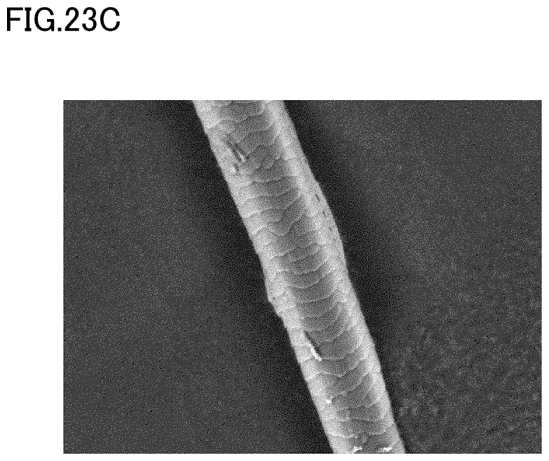

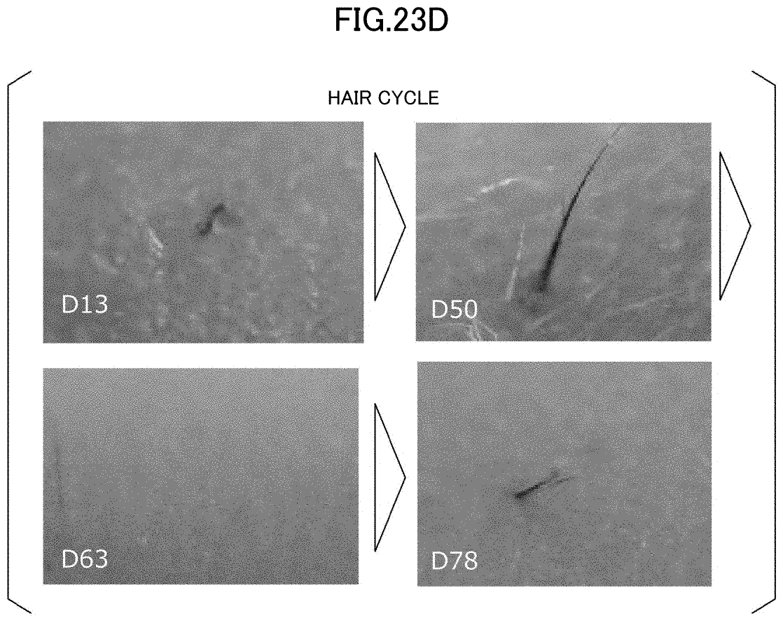

The hair follicle germs obtained in Example 9 were each intradermally transplanted to a nude mouse using the patch method in the same manner as in Test Example 1. The state of regenerated hair at the transplantation portion of the nude mouse on the 16th day from transplantation is shown in FIG. 23A, and the state of regenerated hair at the transplantation portion of the nude mouse after 50 days from transplantation is shown in FIG. 23B.

As shown in FIG. 23A and FIG. 23B, hair regeneration was observed at the transplantation portion transplanted with the hair follicle germs formed using human hair papilla cells as mesenchymal cells.

In addition, an electron micrograph of a hair regenerated at the transplantation portion of the nude mouse after 50 days from transplantation is shown in FIG. 23C. It was confirmed from FIG. 23C that the regenerated hair had a cuticle structure similar to the hair of a human.

Further, the states of regenerated hair at the transplantation portion of the nude mouse until the 13th day, 50th day, 60th day, and 78th day from transplantation are shown in FIG. 23D. It was confirmed from FIG. 23D that, at the transplantation portion, the hair cycle was maintained at least until the 78th day from transplantation.

Thus, it was verified that hair was regenerated even when hair papilla cells derived from the normal scalp of an alopecia patient were used.

### [Example 10] Manufacture of Plurality of Regenerated Mouse Hair Follicle Germs (Using Cell System Derived from Human Alopecia Patient)

1. Production of Microwell Plate

A microwell plate was produced using the same method as in “1.” of Example 1.

2. Formation of Hair Follicle Germs

(1) Preparation of Mesenchymal Cells and Epithelial Cells

With the consent of a human alopecia patient, a normal scalp piece of the human alopecia patient was collected, and hair papilla cells and epidermal keratinocytes were separated from the normal scalp piece and were used as mesenchymal cells and epithelial cells, respectively.

(2) Step of Forming Hair Follicle Germs

Subsequently, a mixed cell suspension of the collected epithelial cells and mesenchymal cells was poured into a poloxamer-treated microwell plate at 1×10⁴ cells/well for each kind of cell (total number of cells: 2×10⁴ cells/well), and the cells were cultured for 3 days. With regard to the composition of a medium used, a medium obtained by mixing DPCGM (manufactured by PromoCell GmbH) and an epithelial cell medium (HuMedia-KG2 medium (manufactured by Kurabo Industries Ltd.)) at 1:1, the medium being supplemented with FGF at 100 ng/mL, was used. The medium was changed every day. After 3 days from the start of culture, it was confirmed that hair follicle germs had been formed.

### [Test Example 6] Subcutaneous Transplantation Test Using Nude Mouse 6

(1) Subcutaneous Transplantation to Nude Mouse

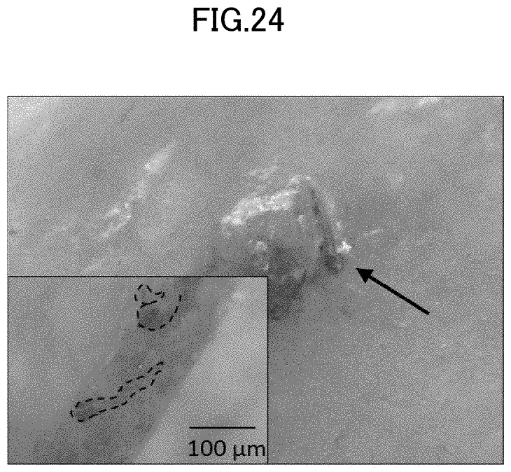

The hair follicle germs obtained in Example 10 were intradermally transplanted to a nude mouse using the patch method in the same manner as in Test Example 1. The state of regenerated hair at the transplantation portion of the nude mouse on the 30th day from transplantation is shown in FIG. 24.

As shown in FIG. 24, hair regeneration was observed at the transplantation portion transplanted with the hair follicle germs formed using human hair papilla cells as mesenchymal cells and human epidermal keratinocytes as epithelial cells.

Thus, it was verified that hair was regenerated even when hair papilla cells and epidermal keratinocytes derived from the normal scalp of an alopecia patient were used.

### INDUSTRIAL APPLICABILITY

According to the manufacturing method for a plurality of regenerated hair follicle germs and the kit for hair regeneration according to the embodiments of the present invention, a plurality of regular and high-density regenerated hair follicle germs having excellent hair regeneration efficiency and similar to hair follicle tissues of a mammal were obtained. In addition, through the use of the manufacturing method for a plurality of regenerated hair follicle germs, a hair follicle tissue-containing sheet including regular and high-density hair follicle tissues having excellent hair regeneration efficiency was manufactured. The resultant hair follicle tissue-containing sheet is suitably used for transplantation to the epidermis of the body, such as a scalp. In addition, through the use of the manufacturing method for a plurality of regenerated hair follicle germs, screening for a hair growth-promoting substance or a hair growth-suppressing substance was performed in a simple manner.

### REFERENCE SIGNS LIST

1 . . . mesenchymal cell, 2 . . . epithelial cell, 3 . . . cell capable of constructing blood vessel, 4 . . . microwell plate, 5 . . . microwell portion, 6, 6b . . . hair follicle germs, 6a . . . mixed spheroid, 7 . . . capillary structure, 8a, 8b . . . medium containing FGF, 9 . . . biocompatible hydrogel, 10 . . . hair follicle tissue-containing sheet

1. A manufacturing method for a plurality of regenerated hair follicle germs, comprising a step including simultaneously inoculating a microwell plate including regularly arranged microwell portions with mesenchymal cells and epithelial cells, and co-culturing the mesenchymal cells and the epithelial cells using a medium containing a fibroblast growth factor while supplying oxygen to the mesenchymal cells and the epithelial cells from at least an upper surface and a bottom surface of the microwell plate, to thereby form hair follicle germs in the microwell portions, the microwell plate being formed of a material having oxygen permeability.

2. The manufacturing method for a plurality of regenerated hair follicle germs according to claim 1, wherein the step of forming hair follicle germs further includes inoculating the microwell plate with cells capable of constructing blood vessels simultaneously with the mesenchymal cells and the epithelial cells.

3. The manufacturing method for a plurality of regenerated hair follicle germs according to claim 2, wherein the cells capable of constructing blood vessels are vascular endothelial cells.

4. The manufacturing method for a plurality of regenerated hair follicle germs according to claim 1, wherein the fibroblast growth factor is basic fibroblast growth factor.

5. The manufacturing method for a plurality of regenerated hair follicle germs according to claim 1, wherein a content of the fibroblast growth factor in the medium is 1 ng/mL or more and 200 ng/mL or less.

6. The manufacturing method for a plurality of regenerated hair follicle germs according to claim 1, wherein the medium contains platelet rich plasma as the fibroblast growth factor.

7. A manufacturing method for a hair follicle tissue-containing sheet, comprising a step of transferring a plurality of regenerated hair follicle germs obtained by the manufacturing method for a plurality of regenerated hair follicle germs of claim 1 to a biocompatible hydrogel in a state in which the plurality of regenerated hair follicle germs are kept in the microwell portions.

8. The manufacturing method for a hair follicle tissue-containing sheet according to claim 7, wherein a density of the microwell portions in the microwell plate is 20 wells/cm² or more and 500 wells/cm² or less.

9. The manufacturing method for a hair follicle tissue-containing sheet according to claim 7, wherein the biocompatible hydrogel is collagen.

10. A kit for hair regeneration, comprising:
a microwell plate including regularly arranged microwell portions; and
a fibroblast growth factor,
wherein the microwell plate is formed of a material having oxygen permeability.

11. The kit for hair regeneration according to claim 10, further comprising a medium.

12. The kit for hair regeneration according to claim 11, wherein the medium is one or more kinds selected from the group consisting of a mesenchymal cell growth medium, an epithelial cell growth medium, and a vascular endothelial cell growth medium.

13. The kit for hair regeneration according to claim 10, wherein the fibroblast growth factor is basic fibroblast growth factor.

14. The kit for hair regeneration according to claim 10, wherein the kit comprises platelet rich plasma as the fibroblast growth factor.

15. A screening method for a hair growth-promoting substance or a hair growth-suppressing substance, comprising:
a step 1 of bringing a candidate substance into contact with mesenchymal cells and epithelial cells;
a step 2 including inoculating a culture vessel with each of: the mesenchymal cells and the epithelial cells brought into contact with the candidate substance; and as a control, mesenchymal cells and epithelial cells free of contact with the candidate substance, and co-culturing the cells using a medium containing a fibroblast growth factor while supplying oxygen, to thereby form hair follicle germs brought into contact with the candidate substance and control hair follicle germs in the culture vessel; and
a step 3 including: judging the candidate substance to be a hair growth-promoting substance when a hair shaft-like structure is formed in the hair follicle germs brought into contact with the candidate substance earlier than in the control hair follicle germs; judging the candidate substance to be a hair growth-suppressing substance when a hair shaft-like structure is formed in the hair follicle germs brought into contact with the candidate substance later than in the control hair follicle germs; and judging the candidate substance to be neither the hair growth-promoting substance nor the hair growth-suppressing substance when a hair shaft-like structure is formed in the hair follicle germs brought into contact with the candidate substance at the same time as in the control germs.
